(19) United States
(12) Patent Application Publication
(10) Pub. No.: US 2025/0099605 A1
(43) Pub. Date: Mar. 27, 2025

(54) TREATMENT OF NON-SMALL CELL LUNG CANCER USING SACITUZUMAB GOVITECAN AND AN ANTI-PD-1 ANTIBODY OR ANTIGEN BINDING FRAGMENT THEREOF

(71) Applicants: Gilead Sciences, Inc., Foster City, CA (US); MSD International GmbH, Luzern (CH); MSD International Business GmbH, Luzern (CH)

(72) Inventors: Sabeen Fatima MEKAN, New York, NY (US); Bilal PIPERDI, Washington, NJ (US); Amy Elizabeth OTTO, Woodinville, WA (US)

(21) Appl. No.: 18/805,405

(22) Filed: Aug. 14, 2024

Related U.S. Application Data
(60) Provisional application No. 63/519,818, filed on Aug. 15, 2023.
(60) Provisional application No. 63/581,239, filed on Sep. 7, 2023.
(60) Provisional application No. 63/592,031, filed on Oct. 20, 2023.
(60) Provisional application No. 63/604,632, filed on Nov. 30, 2023.
(60) Provisional application No. 63/564,626, filed on Mar. 13, 2024.

(51) Int. Cl. A61K 47/68; A61K 39/395; A61P 35/00

(52) U.S. Cl. CPC A61K 47/6851; A61K 39/3955; A61K 47/68037; A61P 35/00

(57) ABSTRACT

The present disclosure relates to methods of treating a treatment naïve metastatic NSCLC in a patient, comprising administering sacituzumab govitecan (SG) and an anti-PD-1 antibody or antigen binding fragment thereof.

### CROSS REFERENCE TO RELATED APPLICATIONS

This application claims the benefit of priority to U.S. Provisional Patent Application No. 63/604,632, filed Nov. 30, 2023, U.S. Provisional Patent Application No. 63/592,031, filed Oct. 20, 2023, U.S. Provisional Patent Application No. 63/581,239, filed Sep. 7, 2023, U.S. Provisional Patent Application No. 63/519,818, filed Aug. 15, 2023, and U.S. Provisional Patent Application No. 63/564,626, filed on Mar. 13, 2024, the entirety of which are incorporated by reference herein.

### INCORPORATION BY REFERENCE OF SEQUENCE LISTING

The instant application contains a Sequence Listing which has been submitted electronically in XML file format and is hereby incorporated by reference in its entirety. Said XML copy, created on Aug. 13, 2024, is named 210196-304007_US_SL.xml and is 19,958 bytes in size.

### FIELD

The present disclosure relates to methods of treatment of non-small cell lung cancer using sacituzumab govitecan (“SG”) and an anti-PD-1 antibody or antigen binding fragment thereof.

### BACKGROUND

Lung cancer (both small cell and non-small cell) is the second most common cancer in both men and women in the U.S. (not counting skin cancer). About 10-15% of all lung cancers are small cell and about 80-85% of all lung cancers are non-small cell (www.cancer.org).

Programmed death (ligand) 1 (PD-[L]1) inhibitor-based regimens are the standard of care in first-line metastatic non-small cell lung cancer (“NSCLC”); however, additional therapies are needed to further improve outcomes.

Sacituzumab govitecan (“SG”) is an antibody-drug-conjugate (“ADC”) comprised of a (a) a humanized monoclonal antibody (“hRS7”) that binds to the cell-surface receptor, Trop 2, (b) a payload (“SN-38”) that is a topoisomerase I inhibitor, and (c) a linker (“CL2A”), that couples the antibody to the payload. There is a high expression of Trop 2 in non-small cell lung cancers.

The high expression of Trop 2 in non-small cell lung cancers, coupled to the high unmet need, led to the design of a phase 2, open-label, multi-cohort, Ph 2 EVOKE-02 study (NCT05186974) evaluating SG+pembrolizumab±platinum agent as first-line treatment for metastatic NSCLC.

SG is marketed under the name TRODELVY and is indicated for (1) adult patients with unresectable locally advanced or metastatic HR+/HER2-breast cancer who have received endocrine-based therapy and at least 2 additional systemic therapies in the metastatic setting; (2) adult patients with unresectable locally advanced or mTNBC who have received 2 or more prior systemic therapies, at least 1 of them for metastatic disease; and (3) adult patients with locally advanced or mUC who have previously received a platinum-containing chemotherapy and either programmed death receptor-1 (PD-1) or programmed death-ligand 1 (PD-L1) inhibitor (accelerated approval only).

PD-1 is recognized as an important molecule in immune regulation and the maintenance of peripheral tolerance. PD-1 is moderately expressed on naive T, B, and NKT cells and up-regulated by T/B cell receptor signaling on lymphocytes, monocytes, and myeloid cells (Sharpe, Arlene H et al., The function of programmed cell death 1 and its ligands in regulating autoimmunity and infection. Nature Immunology (2007); 8:239-245).

Two known ligands for PD-1, PD-L1 (B7-H1) and PD-L2 (B7-DC), are expressed in human cancers arising in various tissues. In large sample sets of cancers, e.g. ovarian, renal, colorectal, pancreatic, liver cancers and melanoma, it was shown that PD-L1 expression correlated with poor prognosis and reduced overall survival irrespective of subsequent treatment (Dong, Haidong et al., Tumor-associated B7-H1 promotes T-cell apoptosis: a potential mechanism of immune evasion. Nat Med. 2002 August; 8 (8): 793-800; Yang, Wanhua et al., PD-1 interaction contributes to the functional suppression of T-cell responses to human uveal melanoma cells in vitro. Invest Ophthalmol Vis Sci. 2008 June; 49 (6 (2008): 49: 2518-2525; Ghebeh, Hazem et al., The B7-H1 (PD-L1) T lymphocyte-inhibitory molecule is expressed in breast cancer patients with infiltrating ductal carcinoma: correlation with important high-risk prognostic factors. Neoplasia (2006) 8:190-198; Hamanishi, Junzo et al., Programmed cell death 1 ligand 1 and tumor-infiltrating CD8+T lymphocytes are prognostic factors of human ovarian cancer. Proc. Natl. Acad. Sci. USA (2007): 104: 3360-3365; Thompson, R Houston, and Eugene D Kwon, Significance of B7-H1 overexpression in kidney cancer. Clinical genitourin Cancer (2006): 5: 206-211; Nomi, Takeo et al., Clinical significance and therapeutic potential of the programmed death-1 ligand/programmed death-1 pathway in human pancreatic cancer. Clinical Cancer Research (2007); 13:2151-2157; Ohigashi, Yuichiro et al., Clinical significance of programmed death-1 ligand-1 and programmed death-1 ligand 2 expression in human esophageal cancer. Clin. Cancer Research (2005): 11: 2947-2953; Inman, Brant A et al., PD-L1 (B7-H1) expression by urothelial carcinoma of the bladder and BCG-induced granulomata: associations with localized stage progression. Cancer (2007): 109: 1499-1505; Shimauchi, Takatoshi et al., Augmented expression of programmed death-1 in both neoplasmatic and nonneoplastic CD4+ T-cells in adult T-cell Leukemia/Lymphoma. Int. J. Cancer (2007): 121:2585-2590; Gao, Qiang et al., Overexpression of PD-L1 significantly associates with tumor aggressiveness and postoperative recurrence in human hepatocellular carcinoma. Clinical Cancer Research (2009) 15:971-979; Nakanishi, Juro et al., Overexpression of B7-H1 (PD-L1) significantly associates with tumor grade and postoperative prognosis in human urothelial cancers. Cancer Immunol Immunother. (2007) 56:1173-1182; Hino et al., Tumor cell expression of programmed cell death-1 is a prognostic factor for malignant melanoma. Cancer (2010): 00: 1-9). Similarly, PD-1 expression on tumor infiltrating lymphocytes was found to mark dysfunctional T cells in breast cancer and melanoma (Ghebeh, Hazem et al., Foxp3+ tregs and B7-H1+/PD-1+T lymphocytes co-infiltrate the tumor tissues of high-risk breast cancer patients: implication for immunotherapy. BMC Cancer. 2008 Feb. 23; 8:57; Ahmadzadeh, Mojgan et al., Tumor antigen-specific CD8 T cells infiltrating the tumor express high levels of PD-1 and are functionally impaired. Blood (2009) 114:1537-1544) and to correlate with poor prognosis in renal cancer (Thompson, R Houston et al., PD-1 is expressed by tumor infiltrating cells and is associated with poor outcome for patients with renal carcinoma. Clinical Cancer Research (2007) 15:1757-1761). Thus, it has been proposed that PD-L1-expressing tumor cells interact with PD-1-expressing T cells to attenuate T cell activation and evasion of immune surveillance, thereby contributing to an impaired immune response against the tumor.

Several monoclonal antibodies that inhibit the interaction between PD-1 and one or both of its ligands PD-L1 and PD-L2 have been approved for treating cancer. Pembrolizumab (KEYTRUDA®, Merck & Co., Inc., Rahway, NJ, USA) is a potent humanized immunoglobulin G4 (IgG4) mAb with high specificity of binding to the programmed cell death 1 (PD-1) receptor, thus inhibiting its interaction with programmed cell death ligand 1 (PD-L1) and programmed cell death ligand 2 (PD-L2). Based on preclinical in vitro data, pembrolizumab has high affinity and potent receptor blocking activity for PD-1. Keytruda® (pembrolizumab) is indicated for the treatment of patients across a number of indications and is indicated for the first-line treatment of patients with unresectable or metastatic CRC that is microsatellite instability-high or mismatch repair deficient (MSI-H/dMMR). Pembrolizumab is the current standard of care for first line MSI-H/dMMR mCRC.

### SUMMARY

One aspect of the present disclosure provides a method of treating a treatment-naïve metastatic non-small cell lung cancer (NSCLC) in a patient, the method comprises: (a) administering 10 mg/kg of sacituzumab govitecan (SG) as an intravenous infusion once weekly on day 1 and day 8 of one or more 21-day treatment cycles, and (b) administering 200 mg of an anti-PD-1 antibody or antigen-binding fragment thereof as an intravenous infusion on day 1 of one or more 21-day treatment cycles, wherein the method achieves a complete response or a partial response in the patient, and wherein the anti-PD-1 antibody or antigen-binding fragment thereof comprises light chain complementarity determining regions (CDRs) comprising a sequence of amino acids as set forth in SEQ ID NOs: 3, 4 and 5 and heavy chain CDRs comprising a sequence of amino acids as set forth in SEQ ID NOs: 8, 9 and 10.

Another aspect of the present disclosure provides a method of treating a metastatic NSCLC in a patient, wherein the patient has not had prior systemic therapy for metastatic NSCLC, the method comprises: (a) administering 10 mg/kg of SG as an intravenous infusion once weekly on day 1 and day 8 of one or more 21-day treatment cycles, and (b) administering 200 mg of an anti-PD-1 antibody or antigen-binding fragment thereof as an intravenous infusion on day 1 of one or more 21-day treatment cycles, wherein the method achieves a complete response or a partial response in the patient, and wherein the anti-PD-1 antibody or antigen-binding fragment thereof comprises light chain complementarity determining regions (CDRs) comprising a sequence of amino acids as set forth in SEQ ID NOs: 3, 4 and 5 and heavy chain CDRs comprising a sequence of amino acids as set forth in SEQ ID NOs: 8, 9 and 10.

In certain embodiments, the patient has a PD-L tumor proportion score of ≥50%.

In certain other embodiments, the patient has a PD-L1 tumor proportion score of ≤50%. In certain embodiments, the patient has a PD-L1 tumor proportion score of 1%-49%. In certain embodiments, the patient has a PD-L1 tumor proportion score of ≤1%.

In certain embodiments, the patient achieves a partial response.

Another aspect of the present disclosure provides a method of treating a treatment-naïve metastatic NSCLC in a patient, wherein the patient has a PD-L1 tumor proportion score of ≥50%, comprising: (a) administering 10 mg/kg of SG as an intravenous infusion once weekly on day 1 and day 8 of one or more 21-day treatment cycles, and (b) administering 200 mg of an anti-PD-1 antibody or antigen-binding fragment thereof as an intravenous infusion on day 1 of one or more 21-day treatment cycles, wherein the method achieves a complete response or a partial response in the patient, and wherein the anti-PD-1 antibody or antigen-binding fragment thereof comprises light chain complementarity determining regions (CDRs) comprising a sequence of amino acids as set forth in SEQ ID NOs: 3, 4 and 5 and heavy chain CDRs comprising a sequence of amino acids as set forth in SEQ ID NOs: 8, 9 and 10.

Another aspect of the present disclosure provides a method of treating metastatic NSCLC in a patient, wherein the patient has a PD-L1 tumor proportion score of ≥50%, and wherein the patient has had no prior systemic treatment for metastatic NSCLC, comprising: (a) administering 10 mg/kg of SG as an intravenous infusion once weekly on day 1 and day 8 of one or more 21-day treatment cycles, and (b) administering 200 mg of an anti-PD-1 antibody or antigen-binding fragment thereof as an intravenous infusion on day 1 of one or more 21-day treatment cycles, wherein the method achieves a complete response or a partial response in the patient, wherein the anti-PD-1 antibody or antigen-binding fragment thereof comprises light chain complementarity determining regions (CDRs) comprising a sequence of amino acids as set forth in SEQ ID NOs: 3, 4 and 5 and heavy chain CDRs comprising a sequence of amino acids as set forth in SEQ ID NOs: 8, 9 and 10.

In certain embodiments, when the method is used to treat metastatic NSCLC in a population of patients, the method achieves an objective response rate of about 75% or greater.

Another aspect of the present disclosure provides a method of treating a treatment-naïve metastatic NSCLC in a patient, wherein the patient has a PD-L1 tumor proportion score of <50%, comprising: (a) administering 10 mg/kg of SG as an intravenous infusion once weekly on day 1 and day 8 of one or more 21-day treatment cycles, and (b) administering 200 mg of an anti-PD-1 antibody, or antigen binding fragment thereof as an intravenous infusion on day 1 of one or more 21-day treatment cycles, wherein the method achieves a complete response or a partial response in the patient, and wherein the anti-PD-1 antibody or antigen-binding fragment thereof comprises light chain complementarity determining regions (CDRs) comprising a sequence of amino acids as set forth in SEQ ID NOs: 3, 4 and 5 and heavy chain CDRs comprising a sequence of amino acids as set forth in SEQ ID NOs: 8, 9 and 10.

Another aspect of the present disclosure further provides a method of treating a metastatic NSCLC in a patient, wherein the patient has a PD-L1 tumor proportion score of <50%, and wherein the patient has had no prior systemic therapy for metastatic NSCLC, comprising: (a) administering 10 mg/kg of SG as an intravenous infusion once weekly on day 1 and day 8 of one or more 21-day treatment cycles, and (b) 200 mg of an anti-PD-1 antibody, or antigen binding fragment thereof as an intravenous infusion on day 1 of one or more 21-day treatment cycles, wherein the method achieves a complete or a partial response in the patient, and wherein the anti-PD-1 antibody or antigen-binding fragment thereof comprises light chain complementarity determining regions (CDRs) comprising a sequence of amino acids as set forth in SEQ ID NOs: 3, 4 and 5 and heavy chain CDRs comprising a sequence of amino acids as set forth in SEQ ID NOs: 8, 9 and 10.

In certain embodiments, when the method is used to treat metastatic NSCLC in a population of patients, the method achieves an objective response rate of about 44%.

Another aspect of the present disclosure further provides a method of achieving a partial response or a complete response, comprising administering to a patient in need thereof: (a) 10 mg/kg of SG as an intravenous infusion once weekly on day 1 and day 8 of one or more 21-day treatment cycles, and (b) 200 mg of an anti-PD-1 antibody, or antigen binding fragment thereof as an intravenous infusion on day 1 of one or more 21-day treatment cycles, wherein the patient has treatment naïve metastatic NSCLC, and wherein the anti-PD-1 antibody or antigen-binding fragment thereof comprises light chain complementarity determining regions (CDRs) comprising a sequence of amino acids as set forth in SEQ ID NOs: 3, 4 and 5 and heavy chain CDRs comprising a sequence of amino acids as set forth in SEQ ID NOs: 8, 9 and 10.

Yet, another aspect of the present disclosure further provides a method of achieving a partial response or a complete response, comprising administering to a patient in need thereof: (a) 10 mg/kg of SG as an intravenous infusion once weekly on day 1 and day 8 of one or more 21-day treatment cycles, and (b) 200 mg of an anti-PD-1 antibody, or antigen binding fragment thereof as an intravenous infusion on day 1 of one or more 21-day treatment cycles, wherein the patient has metastatic NSCLC, wherein the patient has not had prior systemic treatment for metastatic NSCLC, and wherein the anti-PD-1 antibody or antigen-binding fragment thereof comprises light chain complementarity determining regions (CDRs) comprising a sequence of amino acids as set forth in SEQ ID NOs: 3, 4 and 5 and heavy chain CDRs comprising a sequence of amino acids as set forth in SEQ ID NOs: 8, 9 and 10.

In certain embodiment, the patient has a PD-L1 tumor proportion score of ≥50%.

In certain other embodiment, the patient has a PD-L1 tumor proportion score of <50%. In certain embodiments, the patient has a PD-L1 tumor proportion score of 1%-49%. In certain embodiments, the patient has a PD-L1 tumor proportion score of <1%.

In certain embodiment, when the method is used to treat a population of patients having metastatic NSCLC, the objective response rate is about 54%.

Yet, another aspect of the present disclosure further provides a method of achieving an objective response rate of at least 44% in a population of patients having treatment naïve metastatic NSCLC, comprising: (a) administering 10 mg/kg of SG as an intravenous infusion once weekly on day 1 and day 8 of one or more 21-day treatment cycles, and (b) administering 200 mg of an anti-PD-1 antibody, or antigen binding fragment thereof as an intravenous infusion on day 1 of one or more 21-day treatment cycles, wherein the anti-PD-1 antibody or antigen-binding fragment thereof comprises light chain complementarity determining regions (CDRs) comprising a sequence of amino acids as set forth in SEQ ID NOs: 3, 4 and 5 and heavy chain CDRs comprising a sequence of amino acids as set forth in SEQ ID NOs: 8, 9 and 10.

A further aspect of the present disclosure provides a method of achieving an objective response rate of at least 44% in a population of patients having metastatic NSCLC, wherein the patients have not had prior systemic therapy for metastatic NSCLC, comprising: (a) administering 10 mg/kg of SG as an intravenous infusion once weekly on day 1 and day 8 of one or more 21-day treatment cycles, and (b) administering 200 mg of an anti-PD-1 antibody, or antigen binding fragment thereof as an intravenous infusion on day 1 of one or more 21-day treatment cycles, wherein the anti-PD-1 antibody or antigen-binding fragment thereof comprises light chain complementarity determining regions (CDRs) comprising a sequence of amino acids as set forth in SEQ ID NOs: 3, 4 and 5 and heavy chain CDRs comprising a sequence of amino acids as set forth in SEQ ID NOs: 8, 9 and 10.

In certain embodiments, the patients have a PD-L1 tumor proportion score of ≥50%, and wherein the objective response rate is about 75%.

Yet, a further aspect of the present disclosure provides a method of achieving a partial response in a population of patients having metastatic NSCLC, wherein the patients have not had prior systemic therapy for metastatic NSCLC, comprising: (a) administering 10 mg/kg of SG as an intravenous infusion once weekly on day 1 and day 8 of one or more 21-day treatment cycles, and (b) administering 200 mg of an anti-PD-1 antibody, or antigen binding fragment thereof as an intravenous infusion on day 1 of one or more 21-day treatment cycles, wherein the anti-PD-1 antibody or antigen-binding fragment thereof comprises light chain complementarity determining regions (CDRs) comprising a sequence of amino acids as set forth in SEQ ID NOs: 3, 4 and 5 and heavy chain CDRs comprising a sequence of amino acids as set forth in SEQ ID NOs: 8, 9 and 10.

In certain embodiments, the PD-L1 tumor proportion score of the patients is <50%. In certain embodiments, the PD-L1 tumor proportion score of the patients is 1%-49%. In certain embodiments, the PD-L1 tumor proportion score of the patients is <1%.

In certain embodiments, the method does not include administering platinum-based chemotherapy.

In certain embodiments, the patient does not receive platinum-based chemotherapy during the one or more 21-day treatment cycles.

In certain embodiments, the anti-PD-1 antibody, or antigen binding fragment thereof is administered for up to 35 cycles.

In certain embodiments, the patient has non-squamous NSCLC. In certain other embodiments, the patient has squamous NSCLC.

In certain embodiments, the patient has no actionable genomic alterations in EGFR, ALK, and PD-L1.

In certain embodiments, the patient has no known actionable genomic alterations in ROS1, NTRK, BRAF, and RET.

In certain embodiments, the patient does not have actionable genomic alterations in EGFR and ALK.

In certain embodiments, the patient does not receive carboplatin, paclitaxel, or paclitaxel protein-bound during the one or more 21-day treatment cycles.

In certain embodiments, when the method is used to treat a population of patients having metastatic NSCLC across all PD-L1 scores, the disease control rate (DCR) is about 81% after at least 6 weeks.

In certain embodiments, when the method is used to treat a population of patients having metastatic NSCLC and a PD-L1 tumor proportion scores of ≥50%, the disease control rate (DCR) is greater than or equal to about 88% after at least 6 weeks.

In certain embodiments, when the method is used to treat a population of patients having metastatic NSCLC and a PD-L1 tumor proportion scores of <50%, the disease control rate (DCR) is greater than or equal to about 78% after at least 6 weeks.

In certain embodiments, when the method is used to treat a population of patients having metastatic NSCLC, the duration of response (DOR) is about 88% at about 6 months.

In certain embodiments, then patient has stage IV metastatic NSCLC.

In certain embodiments, the method is used to treat metastatic NSCLC in a population of patients, the method achieves less than about 10% serious treatment emergent adverse effects (TEAEs).

In certain embodiments, the method is used to treat metastatic NSCLC in a population of patients, the method achieves less than about 9% serious TEAEs.

In certain embodiments, the method is used to treat metastatic NSCLC in a population of patients, the method achieves less than about 25% TEAEs of grade≥3.

In certain embodiments, the method is used to treat metastatic NSCLC in a population of patients, the method achieves less than about 24% TEAEs of grade≥3.

In certain embodiments, the anti-PD-1 antibody or antigen-binding fragment thereof comprises a light chain variable region comprising SEQ ID NO: 6 or a variant thereof, and a heavy chain variable region comprising SEQ ID NO: 11.

In certain embodiments, the anti-PD-1 antibody or antigen-binding fragment thereof comprises a light chain comprising SEQ ID NO: 7 and a heavy chain comprising SEQ ID NO: 12.

In certain embodiments, the anti-PD-1 antibody or antigen-binding fragment thereof is pembrolizumab or a variant thereof.

In certain specific embodiments, the anti-PD-1 antibody or antigen-binding fragment thereof is pembrolizumab.

Another aspect of the present disclosure further provides use of a therapeutic combination comprising Sacituzumab govitecan (SG) and an anti-PD-1 antibody or antigen binding fragment thereof for the method described herein.

A further aspect of the present disclosure provides a therapeutic combination comprising Sacituzumab govitecan (SG) and an anti-PD-1 antibody or antigen binding fragment thereof for use in the method described herein.

In certain embodiments, the method does not include administering an anti-TIGIT antibody.

In certain embodiments, the patient does not receive an anti-TIGIT antibody during the one or more 21-day treatment cycles.

In certain embodiments, disclosed herein are methods of treating a NSCLC in a patient, wherein the patient has not had prior systemic therapy for metastatic NSCLC, comprising co-administering: (a) 10 mg/kg of an antibody-drug conjugate (ADC) comprising an anti-Trop-2 antibody or an antigen-binding fragment thereof as an intravenous infusion on day 1 and day 8 of one or more 21-day treatment cycles, wherein the anti-Trop-2 antibody or the antigen binding fragment thereof comprises light chain complementarity determining regions (CDRs) comprising a sequence of amino acids as set forth in SEQ ID NOs: 13, 14 and 15 and heavy chain CDRs comprising a sequence of amino acids as set forth in SEQ ID NOs: 16, 17 and 18, and wherein the ADC has a formula MAb-CL2A-SN-38, with the formula represented by:

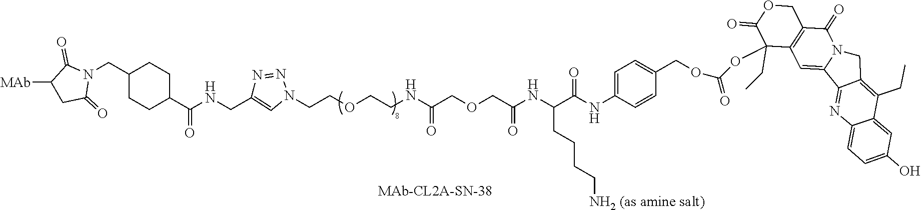

and (b) 200 mg of an anti-PD-1 antibody or an antigen-binding fragment thereof as an intravenous infusion on day 1 of the one or more 21-day treatment cycles, wherein the anti-PD-1 antibody or the antigen-binding fragment thereof comprises light chain CDRs comprising a sequence of amino acids as set forth in SEQ ID NOs: 3, 4 and 5 and heavy chain CDRs comprising a sequence of amino acids as set forth in SEQ ID NOs: 8, 9 and 10; and wherein the method achieves a complete response or a partial response in the patient.

In certain embodiments, the method does not comprise administration of an additional and/or a third anti-cancer agent. In certain embodiments, the patient maintains a partial response at least about 13 weeks or greater from baseline. In certain embodiments, the patient has a PD-L1 TPS score of <50%, and the partial response is about 60% or greater reduction in the size of one or more measurable tumors from baseline to approximately 13 weeks or more from baseline. In certain embodiments, the patient has a PD-L1 TPS of <1%, and the partial response is about 50% or greater reduction in the size of one or more measurable tumors from baseline to approximately 13 weeks or more from baseline. In certain embodiments, the patient has a PD-L1 TPS of 1%-49%, and the partial response is about 60% or greater reduction in the size of one or more measurable tumors from baseline to approximately 13 weeks or more from baseline. In certain embodiments, the patient has no actionable genetic alterations in EGFR, ALK, and PD-L1. In certain embodiments, the patient has no known actionable genetic alterations in ROS1, NTRK, BRAF, and RET. In certain embodiments, the patient has no actionable genetic alterations in EGFR and ALK. In certain embodiments, the patient has not had prior targeted therapy for metastatic NSCLC. In certain embodiments, the patient has not had prior chemotherapy for metastatic NSCLC. In certain embodiments, the patient maintains a partial response from baseline to at least approximately 48 weeks from baseline.

In certain embodiments, the anti-Trop-2 antibody or the antigen-binding fragment thereof comprises a heavy chain variable region comprising SEQ ID NO: 19, and a light chain variable region comprising SEQ ID NO: 20.

In certain embodiments, the anti-Trop-2 antibody comprises a heavy chain comprising SEQ ID NO: 1, and a light chain comprising SEQ ID NO: 2.

In certain embodiments, the anti-Trop-2 antibody is sacituzumab.

In certain further embodiments, the ADC is sacituzumab govitecan (SG).

In certain embodiments, the anti-PD-1 antibody or the antigen-binding fragment thereof comprises a light chain variable region comprising SEQ ID NO: 6 or a variant thereof, and a heavy chain variable region comprising SEQ ID NO: 11.

In certain embodiments, the anti-PD-1 antibody or the antigen-binding fragment thereof comprises a light chain comprising SEQ ID NO: 7 and a heavy chain comprising SEQ ID NO: 12.

In certain embodiments, the anti-PD-1 antibody or the antigen-binding fragment thereof is pembrolizumab or a variant thereof.

In certain embodiments, the PD-1 antibody or the antigen fragment thereof is pembrolizumab.

In certain embodiments, the ADC is SG and the PD-1 antibody, or the antigen fragment thereof, is pembrolizumab.

In certain embodiments, the patient has a PD-L1 tumor proportion score (TPS) of ≥50%. In certain other embodiments, the patient has a PD-L1 TPS of <50%.

In certain embodiments, the patient has non-squamous NSCLC. In certain other embodiments, the patient has squamous NSCLC.

In certain embodiments, the method does not comprise concomitant administration of chemotherapy. In certain embodiments, the method does not comprise concomitant administration of platinum-based chemotherapy. In certain embodiments, the method does not comprise concomitant administration of an anti-TGIT antibody.

In certain embodiments, the partial response is about 50% or greater reduction in a size of one or more measurable tumors from baseline to approximately 13 weeks or more from baseline. In certain embodiments, the partial response is about 60% or greater reduction in the size of one or more of the measurable tumors from baseline to approximately 13 weeks or more from baseline. In certain embodiments, the patient has a PD-L1 TPS of 1%-49%. In certain embodiments, the patient has a PD-L1 TPS of <1%.

In certain embodiments, when the method is used to treat metastatic NSCLC in a population of patients with a PD-L1 TPS of ≥50%, the method achieves a partial response rate of about 69% or greater. In certain other embodiments, when the method is used to treat metastatic NSCLC in a population of patients with a PD-L1 TPS of <50%, the method achieves a partial response rate of about 44% or greater.

In certain embodiments, when the method is used to treat metastatic NSCLC in a population of patients with a PD-L1 TPS of 1%-49%, the method achieves an ORR of about 53%. In certain embodiments, the method achieves a partial response of about 47%-53%. In certain embodiments, the method achieves a partial response of about 47%. In certain embodiments, the method achieves a partial response of about 53%.

In certain embodiments, when the method is used to treat metastatic NSCLC in a population of patients with a PD-L1 TPS of <1%, the method achieves an ORR of about 35%. In certain embodiments, the method achieves a partial response of about 29%-35%. In certain embodiments, the method achieves a partial response of about 29%. In certain embodiments, the method achieves a partial response of about 35%.

In certain embodiments, when the method is used to treat metastatic NSCLC in a population of patients, the method achieves a partial response of about 56% or greater irrespective of the population of patient's PD-L1 TPS status.

In certain embodiments, the method does not include administration of an additional targeted oncology drug.

In certain embodiments, disclosed herein are methods of treating a population of patients having metastatic NSCLC without concomitant administration of chemotherapy comprising co-administering: (a) 10 mg/kg of an antibody-drug conjugate (ADC) comprising an anti-Trop-2 antibody or an antigen-binding fragment thereof as an intravenous infusion on day 1 and day 8 of one or more 21-day treatment cycles, wherein the anti-Trop-2 antibody or the antigen binding fragment thereof comprises light chain complementarity determining regions (CDRs) comprising a sequence of amino acids as set forth in SEQ ID NOs: 13, 14 and 15 and heavy chain CDRs comprising a sequence of amino acids as set forth in SEQ ID NOs: 16, 17 and 18, and wherein the ADC has a formula MAb-CL2A-SN-38, with the formula represented by:

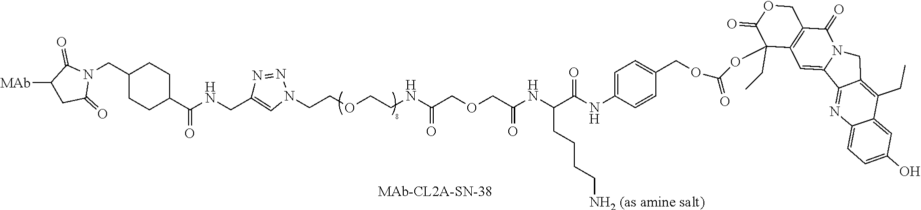

and (b) 200 mg of an anti-PD-1 antibody or an antigen-binding fragment thereof as an intravenous infusion on day 1 of the one or more 21-day treatment cycles, wherein the anti-PD-1 antibody or the antigen-binding fragment thereof comprises light CDRs comprising a sequence of amino acids as set forth in SEQ ID NOs: 3, 4 and 5 and heavy chain CDRs comprising a sequence of amino acids as set forth in SEQ ID NOs: 8, 9 and 10; wherein the population of patients have not received prior systemic therapy for metastatic NSCLC.

In certain embodiments, the population of patients has no actionable genetic alterations in EGFR, ALK, and PD-L1. In certain embodiments, the population of patients has no known actionable genetic alterations in ROS1, NTRK, BRAF, and RET. In certain embodiments, the population of patient has no actionable genetic alterations in EGFR and ALK. In certain embodiments, the population of patients maintain a partial response from baseline to at least approximately 48 weeks from baseline.

In certain embodiments, the anti-Trop-2 antibody or the antigen-binding fragment thereof comprises a heavy chain variable region comprising SEQ ID NO: 19, and a light chain variable region comprising SEQ ID NO: 20.

In certain embodiments, the anti-Trop-2 antibody comprises a heavy chain comprising SEQ ID NO: 1, and a light chain comprising SEQ ID NO: 2.

In certain embodiments, the anti-Trop-2 antibody is sacituzumab.

In certain embodiments, the ADC is SG.

In certain embodiments, the anti-PD-1 antibody or the antigen-binding fragment thereof comprises a light chain variable region comprising SEQ ID NO: 6 or a variant thereof, and a heavy chain variable region comprising SEQ ID NO: 11.

In certain embodiments, the anti-PD-1 antibody or the antigen-binding fragment thereof comprises a light chain comprising SEQ ID NO: 7 and a heavy chain comprising SEQ ID NO: 12.

In certain embodiments, the anti-PD-1 antibody or the antigen-binding fragment thereof is pembrolizumab or a variant thereof. In certain further embodiments, the anti-PD-1 antibody or the antigen-binding fragment thereof is pembrolizumab.

In certain embodiments, the PD-1 antibody or the antigen-binding fragment thereof is pembrolizumab and the ADC is SG.

In certain embodiments, the population of patients has a PD-L1 TPS of ≥50%. In certain other embodiments, the population of patients has a PD-L1 TPS of <50%. In certain embodiments, the population of patients has a PD-L1 TPS of 1%-49%. In certain embodiments, the population of patients has a PD-L1 TPS of <1%.

In certain embodiments, the population of patients has non-squamous NSCLC. In certain other embodiments, the population of patients has squamous NSCLC.

In certain embodiments, the treatment comprises achieving of clinical efficacy.

In certain embodiments, the ORR is about 69%. In certain other embodiments, the ORR is about 44%.

In certain embodiments, the DCR is about 86%. In certain other embodiments, the DCR is about 78%.

In certain embodiments, the DOR at about 6 months from baseline is about 88%.

In certain embodiments, the method achieves less than about 10% serious treatment emergent adverse events (TEAEs). In certain further embodiments, the method achieves less than about 9% serious TEAEs.

In certain embodiments, the method achieves less than about 25% TEAEs of grade≥3. In certain further embodiments, the method achieves less than about 24% TEAEs of grade≥3.

In certain embodiments, the TEAEs comprise one or more of stomatitis, anemia, leukopenia, rash, and thrombocytopenia.

In certain embodiments, a TEAE comprises interstitial lung disease (ILD).

In certain embodiments, ILD is observed in less than about 10% of the patients. In certain further embodiments, ILD is observed in less than about 9% of the patients. In certain further embodiments, ILD is observed in less than about 5% of the patients.

In certain embodiments, ILD is not observed in the patients.

In certain embodiments, the method does not comprise concomitant administration of platinum-based chemotherapy. In certain embodiments, the method does not comprise concomitant administration of platinum-based chemotherapy. In certain embodiments, the method does not comprise concomitant administration of an anti-TGIT antibody.

In certain embodiments, when the population of patients have a PD-L1 TPS of ≥50%, the method achieves a partial response rate of about 69% or greater.

In certain embodiments, when the population of patients have a PD-L1 TPS of <50%, the method achieves a partial response rate of about 44% or greater.

In certain embodiments, the method achieves a partial response of about 56% or greater irrespective of the population of patient's PD-L1 TPS status.

In certain embodiments, the method does not comprise determination of a PD-L1 TPS status in the population of patients.

In certain embodiments, the method does not include administration of an additional targeted oncology drug.

In certain embodiments, the partial response is about 30% or greater reduction in the size of one or more measurable tumors from baseline to approximately 13 weeks or more from baseline.

In certain embodiments, the partial response is about 40% or greater reduction in the size of one or more measurable tumors from baseline to approximately 13 weeks or more from baseline.

In certain embodiments, disclosed herein are methods of improving clinical efficacy compared to administration of an anti-PD-L1 antibody monotherapy or compared to administration of an antibody drug conjugate monotherapy, in a population of patients having metastatic non-squamous or squamous NSCLC, without concomitant administration of chemotherapy, comprising co-administering: (a) 10 mg/kg SG as an intravenous infusion on day 1 and day 8 of one or more 21-day treatment cycles; and (b) 200 mg pembrolizumab on day 1 of the one or more 21-day treatment cycles; wherein the population of patients have not received prior systemic therapy for metastatic NSCLC.

In certain embodiments, the population of patients have no actionable genetic alterations in EGFR, ALK, and PD-L1. In certain embodiments, the patient has no known actionable genetic alterations in ROS1, NTRK, BRAF, and RET. In certain embodiments, the patient has no actionable genetic alterations in EGFR and ALK. In certain embodiments, the population of patients maintain a partial response from baseline to at least approximately 48 weeks from baseline.

In certain embodiments, the anti-PD-1 antibody comprises a light chain variable region comprising SEQ ID NO: 6 or a variant thereof, and a heavy chain variable region comprising SEQ ID NO: 11.

In certain embodiments, the anti-PD-1 antibody or the antigen-binding fragment thereof comprises a light chain comprising SEQ ID NO: 7 and a heavy chain comprising SEQ ID NO: 12.

In certain embodiments, the clinical efficacy is improved irrespective of the population of patient's PD-L1 status.

In certain embodiments, the PD-L1 TPS score of the population of patients is ≥50%. In certain other embodiments, the PD-L1 TPS score of the population of patients is <50%.

In certain embodiments, the anti-PD-L1 antibody monotherapy is pembrolizumab.

In certain embodiments, the antibody drug conjugate monotherapy is SG.

In certain embodiments, the method does not comprise determination of a PD-L1 TPS status in the population of patients. In certain embodiments, the method does not include administration of an additional targeted oncology drug.

In certain embodiments, the chemotherapy is platinum-based chemotherapy.

In certain embodiments, the patient has non-squamous NSCLC. In certain other embodiments, the patient has squamous NSCLC.

In certain embodiments, the method does not comprise concomitant administration of an anti-TGIT antibody.

In certain embodiments, when the population of patients have a PD-L1 TPS of ≥50%, the method achieves a partial response rate of about 69% or greater.

In certain embodiments, when the population of patients have a PD-L1 TPS of <50%, the method achieves a partial response rate of about 44% or greater.

In certain embodiments, the method achieves a partial response of about 56% or greater irrespective of the population of patient's PD-L1 TPS status.

In certain embodiments, the ORR is about 69%. In certain other embodiments, the ORR is about 44%.

In certain embodiments, the DCR is about 86%. In certain other embodiments, the DCR is about 78%.

In certain embodiments, the DOR at about 6 months from baseline is about 88%.

In certain embodiments, the method achieves less than about 10% serious treatment emergent adverse events (TEAEs). In certain further embodiments, the method achieves less than about 9% serious TEAEs.

In certain embodiments, the method achieves less than about 25% TEAEs of grade≥3. In certain further embodiments, the method achieves less than about 24% TEAEs of grade≥3.

In certain embodiments, the TEAEs comprise one or more of stomatitis, anemia, leukopenia, rash, and thrombocytopenia.

In certain embodiments, a TEAE comprises interstitial lung disease (ILD).

In certain embodiments, ILD is observed in less than about 10% of the patients. In certain further embodiments, ILD is observed in less than about 9% of the patients. In certain further embodiments, ILD is observed in less than about 5% of the patients.

In certain embodiments, ILD is not observed in the patients.

In certain embodiments, the partial response is about 30% or greater reduction in the size of one or more measurable tumors from baseline to approximately 13 weeks or more from baseline.

In certain embodiments, the partial response is about 40% or greater reduction in the size of one or more measurable tumors from baseline to approximately 13 weeks or more from baseline.

In certain embodiments, disclosed herein are methods of achieving clinical efficacy in a patient having metastatic NSCLC irrespective of the patient's PD-L1 TPS score, irrespective of the patient's NSCLC histology, without concomitant administration of chemotherapy, and wherein the patient has not received prior systemic therapy for metastatic NSCLC comprising co-administering: (a) 10 mg/kg of SG on day 1 and day 8 of one or more 21-day treatment cycles; and (b) 200 mg of pembrolizumab on day 1 of the one or more 21-day treatment cycles.

In certain embodiments, the PD-L1 TPS score of the population of patients is ≥50%. In certain other embodiments, the PD-L1 TPS score of the population of patients is <50%. In certain embodiments, the PD-L1 TPS of the population of patients is 1%-49%. In certain embodiments, the PD-L1 TPS of the population of patients is <1%.

In certain embodiments, the method does not comprise determination of a PD-L1 TPS status in the population of patients. In certain embodiments, the method does not include administration of an additional targeted oncology drug.

In certain embodiments, the chemotherapy is platinum-based chemotherapy.

In certain embodiments, the patient has non-squamous NSCLC. In certain other embodiments, the patient has squamous NSCLC.

In certain embodiments, the method does not comprise concomitant administration of an anti-TGIT antibody.

In certain embodiments, when the population of patients have a PD-L1 TPS of ≥50%, the method achieves a partial response rate of about 69% or greater.

In certain embodiments, when the population of patients have a PD-L1 TPS of <50%, the method achieves a partial response rate of about 44% or greater.

In certain embodiments, the method achieves a partial response of about 56% or greater irrespective of the population of patient's PD-L1 TPS status.

In certain embodiments, the ORR is about 69%. In certain other embodiments, the ORR is about 44%.

In certain embodiments, the DCR is about 86%. In certain other embodiments, the DCR is about 78%.

In certain embodiments, the DOR at about 6 months from baseline is about 88%.

In certain embodiments, the method achieves less than about 10% serious treatment emergent adverse events (TEAEs). In certain further embodiments, the method achieves less than about 9% serious TEAEs.

In certain embodiments, the method achieves less than about 25% TEAEs of grade≥3. In certain further embodiments, the method achieves less than about 24% TEAEs of grade≥3.

In certain embodiments, the TEAEs comprise one or more of stomatitis, anemia, leukopenia, rash, and thrombocytopenia.

In certain embodiments, a TEAE comprises interstitial lung disease (ILD).

In certain embodiments, ILD is observed in less than about 10% of the patients. In certain further embodiments, ILD is observed in less than about 9% of the patients. In certain further embodiments, ILD is observed in less than about 5% of the patients.

In certain embodiments, ILD is not observed in the patients.

In certain embodiments, the partial response is about 30% or greater reduction in the size of one or more measurable tumors from baseline to approximately 13 weeks or more from baseline.

In certain embodiments, the partial response is about 40% or greater reduction in the size of one or more measurable tumors from baseline to approximately 13 weeks or more from baseline.

In certain embodiments, the method does not comprise concomitant administration of an additional and/or third anti-cancer agent. In certain embodiments, the method does not comprise determination of the patient's PD-L1 TPS status. In certain embodiments, the patient maintains a partial response from baseline to at least approximately 48 weeks from baseline.

In certain embodiments, when the method is used to treat a population of patients having metastatic NSCLC, and clinical efficacy comprises an objective response rate, and objective response rate (ORR) of about 56% or greater is achieved. In certain embodiments, the ORR of about 56% or greater is achieved approximately 13 weeks or greater from baseline.

In certain embodiments, the method does not comprise concomitant administration of an additional targeted oncology drug.

In certain embodiments, disclosed herein are uses of a therapeutic combination comprising the antibody-drug conjugate (ADC) comprising the anti-Trop-2 antibody or the antigen-binding fragment thereof and the anti-PD-1 antibody or the antigen binding fragment thereof for the methods described herein.

In certain embodiments, disclosed herein are therapeutic combinations comprising an antibody-drug conjugate (ADC) comprising an anti-Trop-2 antibody or an antigen-binding fragment thereof and an anti-PD-1 antibody or an antigen binding fragment thereof for use in the methods described herein.

In certain embodiments, the partial response is about 30% or greater reduction in the size of one or more measurable tumors from baseline to approximately 13 weeks or more from baseline.

In certain embodiments, the partial response is about 40% or greater reduction in the size of one or more measurable tumors from baseline to approximately 13 weeks or more from baseline.

In certain embodiments, a method of achieving clinical efficacy in a patient having metastatic NSCLC irrespective of the patient's PD-L1 TPS, irrespective of the patient's NSCLC histology, without the concomitant administration of chemotherapy, wherein the patient has not received prior systemic therapy for metastatic NSCLC, comprising co-administering, 10 mg/kg of SG on day 1 and day 8 of one or more 21-day treatment cycles, and 200 mg of pembrolizumab on day 1 of the one or more 21-day treatment cycles, is provided. In certain embodiments, the patient has a PD-L1 TPS of <1%. In certain embodiments, the method does not comprise concomitant administration of an additional targeted oncology drug.

In certain embodiments, disclosed herein is method of treating a squamous metastatic non-small cell lung cancer (NSCLC) in a patient, wherein the patient has not had a prior systemic therapy for squamous metastatic NSCLC, comprising co-administering: a) 10 mg/kg of SG on day 1 and day 8 of one or more 21-day treatment cycles; and b) 200 mg of pembrolizumab on day 1 of the one or more 21-day treatment cycles.

In certain embodiments, the patient has a PD-L1 tumor proportion score (TPS) of ≥50%. In certain embodiments, the patient has a PD-L1 TPS of <50%.

In certain embodiments, the method does not comprise concomitant administration of chemotherapy. In certain embodiments, the method does not comprise concomitant administration of platinum-based chemotherapy.

In certain embodiments, the method is used to treat squamous metastatic NSCLC in a population of patients with a PD-L1 TPS of ≥50%, the method achieves a partial response rate of about 73% or greater. In certain embodiments, the method is used to treat squamous metastatic NSCLC in a population of patients with a PD-L1 TPS of <50%, the method achieves a partial response rate of about 54% or greater.

In certain embodiments, provided herein is a method of treating a population of patients having squamous metastatic NSCLC without concomitant administration of chemotherapy comprising co-administering: 10 mg/kg of SG on day 1 and day 8 of one or more 21-day treatment cycles; and 200 mg of pembrolizumab on day 1 of the one or more 21-day treatment cycles; wherein the population of patients have not received prior systemic therapy for squamous metastatic NSCLC.

In certain embodiments, the population of patients has a PD-L1 TPS of ≥50%. In certain embodiments, the population of patients has a PD-L1 TPS of <50%.

In certain embodiments, the treatment comprises achieving of clinical efficacy.

In certain embodiments, the ORR is about 73%. In certain other embodiments, the ORR is about 54%.

In certain embodiments, the DCR is about 82%. In certain other embodiments, the DCR is about 85%.

In certain embodiments, the method achieves a partial response of about 64%-73%. In certain other embodiments, the method achieves a partial response of about 46%-57%.

In certain embodiments, provided herein is a method of achieving clinical efficacy in a patient having metastatic squamous NSCLC irrespective of the patient's PD-L1 tumor proportion score, without the concomitant administration of chemotherapy, and wherein the patient has not received prior systemic therapy for metastatic NSCLC, comprising co-administering: 10 mg/kg of SG on day 1 and day 8 of one or more 21-day treatment cycles; and 200 mg of pembrolizumab on day 1 of the one or more 21-day treatment cycles.

In certain embodiments, the method does not comprise concomitant administration of an additional targeted oncology drug.

In certain embodiments, the ORR is about 73%. In certain other embodiments, the ORR is about 54%.

In certain embodiments, the DCR is about 82%. In certain other embodiments, the DCR is about 85%.

In certain embodiments, the method achieves a partial response of about 64%-73%. In certain other embodiments, the method achieves a partial response of about 46%-57%.

In certain embodiments, provided herein is a method of achieving clinical efficacy in a patient having metastatic squamous NSCLC, comprising administering to the patient in need thereof: 10 mg/kg of SG on day 1 and day 8 of one or more 21-day treatment cycles; and 200 mg of pembrolizumab on day 1 of the one more 21-day treatment cycles, wherein the patient's PD-L1 tumor proportion score is ≥50%.

In certain embodiments, the methods disclosed herein comprise administering SG for at least about (12) 21-day treatment cycles. In certain embodiments, the methods disclosed herein comprise administering SG and pembrolizumab for at least about (12) 21-day cycles. In certain embodiments, the patient achieves a partial response for at least about 36 weeks. In certain embodiments, the methods disclosed herein comprise administering SG on day 1 and day 8 of at least about (12) 21-day treatment cycles and administering pembrolizumab on day 1 of at least about (12) 21-day treatment cycles.

In certain embodiments, the methods disclosed herein comprise administering SG for at least about (14) 21-day treatment cycles. In certain embodiments, the methods disclosed herein comprise administering SG and pembrolizumab for at least about (14) 21-day treatment cycles. In certain embodiments, the patient achieves a partial response for at least about 42 weeks. In certain embodiments, the methods disclosed herein comprise administering SG on day 1 and day 8 of at least about (14) 21-day treatment cycles and administering pembrolizumab on day 1 of at least about (14) 21-day treatment cycles.

### BRIEF DESCRIPTION OF THE DRAWINGS

FIG. 1 shows the design of an Open-Label, Multi-Cohort Phase 2 clinical trial (NCT05186974) for evaluating sacituzumab govitecan (SG) in combination with pembrolizumab (pembro), with or without a platinum agent, as first-line treatment for metastatic non-small cell lung cancer (NSCLC). The clinical trial is referred to as “EVOKE-02.” As used in the figure, NSCLC, non-small cell lung cancer; RECIST, Response Evaluation Criteria in Solid Tumors; ECOG, Eastern Cooperative Oncology Group performance score; PD-L1, programmed death-ligand 1; TPS, tumor proportion score; Sq, squamous; Nsq, non-squamous; IV, intravenously; RP2D, recommended phase 2 dose; ORR, objective response rate; DLT, dose-limiting toxicity; DCR, disease control rate; DOR, duration of response; PFS, progression-free survival; and OS, overall survival.

FIGS. 2A-2B show the sequences of the heavy (FIG. 2a) and the light (FIG. 2b) chains of the antibody (hRS7) in SG. FIG. 2A discloses SEQ ID NO: 1. FIG. 2B discloses SEQ ID NO: 2.

FIG. 3 shows Study Procedures for EVOKE-02. As used in the figure: SG, sacituzumab govitecan; EGFR, epithelial growth factor receptor; ALK, anaplastic lymphoma kinase; PD-L1, programmed death-ligand 1PK, pharmacokinetic; CT, computed tomography; MRI, magnetic resonance imaging; 18F-FDG PET, fluorodeoxyglucose F 18 positron emission tomography; AEs, adverse events; and SAEs, serious adverse events. As of data cutoff (Jun. 16, 2023), median (range) follow-up for Cohorts A and B was 5.0 (1.7-12.0) and 5.8 (1.0-12.2) months, respectively.

FIG. 4 shows exemplary data for the best percent reduction in target lesions for patients in EVOKE-02. Each bar represents a patient. The percent reduction in target lesions is shown for each patient in Cohort A or Cohort B of EVOKE-02. The best overall response for each patient is indicated, and each patient is classified as having a best overall response corresponding to a partial response (triangle), a stable disease (square) or a progressive disease (circle). Only patients with ≥13 weeks of follow-up as of the cutoff date (Jun. 16, 2023) were included in the efficacy analysis. As used in the figure: TPS, tumor proportion score.

FIG. 5A-5B show exemplary data on the response from treatment initiation to progression of patients in Cohort A (FIG. 5A) and Cohort B (FIG. 5B) of EVOKE-02. The data shows the percentage change in the measurement of target lesions from baseline as a function of time. Each line corresponds to the response progression of an individual patient, and each dot represents a response assessment timepoint. The last assessment of each patient before data collection is indicated with a circle. The best overall response for each patient is indicated, and each patient is classified as having a best overall response corresponding to a partial response (green lines), a stable disease (blue lines) or a progressive disease (red lines). Only patients with ≥13 weeks of follow-up as of the cutoff date (Jun. 16, 2023) were included in the efficacy analysis. As used in the figure: TPS, tumor proportion score; PD-L1, programmed death-ligand; SG, Sacituzumab govitecan; and pembro, pembrolizumab.

FIG. 6 shows exemplary data on treatment-emergent adverse events (TEAEs) reported for patients in EVOKE-02. Each bar indicates the percent of patients out of the total number of safety-evaluable patients in Cohorts A and B that reportedly had the corresponding TEAE. Only patients that received≥1 dose of study treatment were included in the analysis. For each TEAE, the number of patients having the TEAE at a Grade 1-2, or a Grade 3 or higher are indicated.

FIG. 7 shows exemplary data on immune-mediated TEAEs reported for patients in EVOKE-02. Each bar indicates the percent of patients out of the total number of safety-evaluable patients in Cohorts A and B that reportedly had the TEAE. Only patients that received≥1 dose of study treatment were included in the analysis. For each TEAE, the number of patients having the TEAE at a Grade 1-2, or a Grade 3 or higher are indicated.

### DETAILED DESCRIPTION

Anti-TROP2 ADC: Sacituzumab govitecan (“SG”)

SG is a pharmaceutical composition comprising an antibody-drug conjugate (“ADC”) comprised of (1) a drug (“SN-38”), a topoisomerase 1 inhibitor that is an active metabolite of irinotecan; (2) a linker (“CL2A”); and (3) a humanized monoclonal antibody (“hRS7 IgGk” or “sacituzumab”). CL2A couples SN-38 to hRS7, which binds to Trop-2.

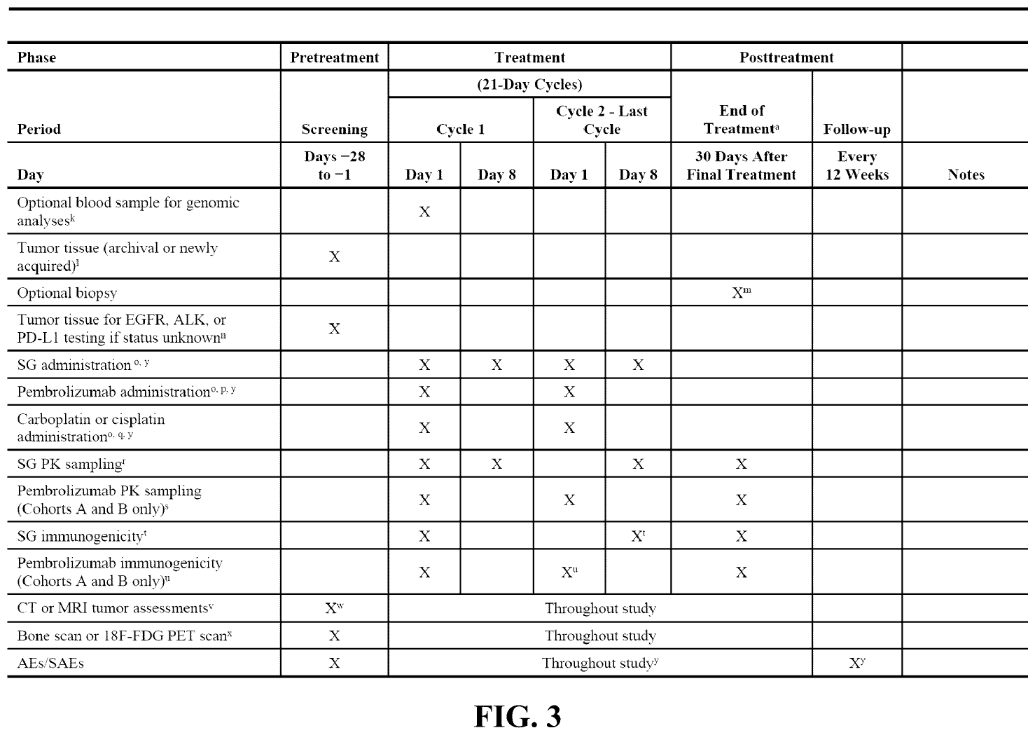

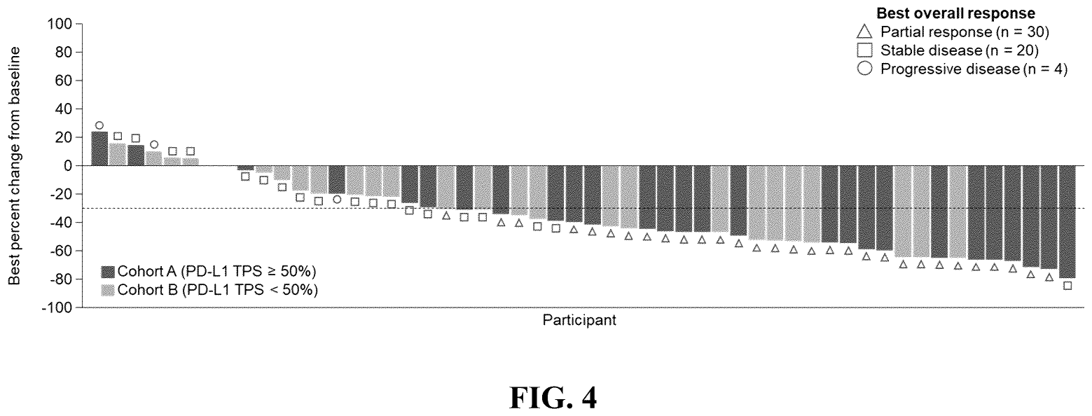

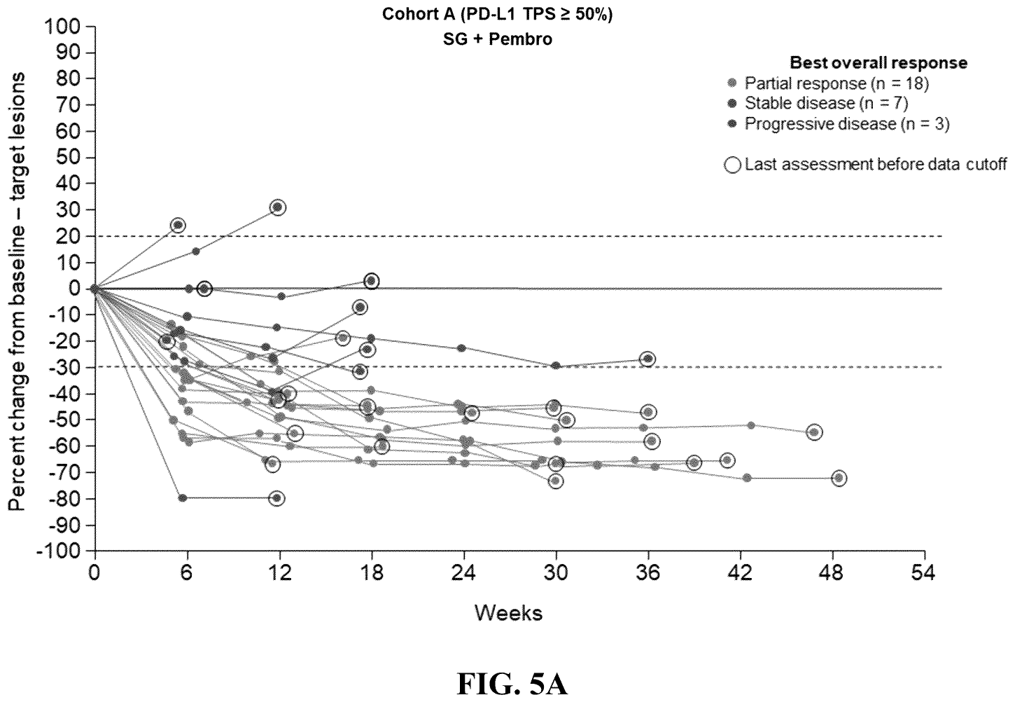

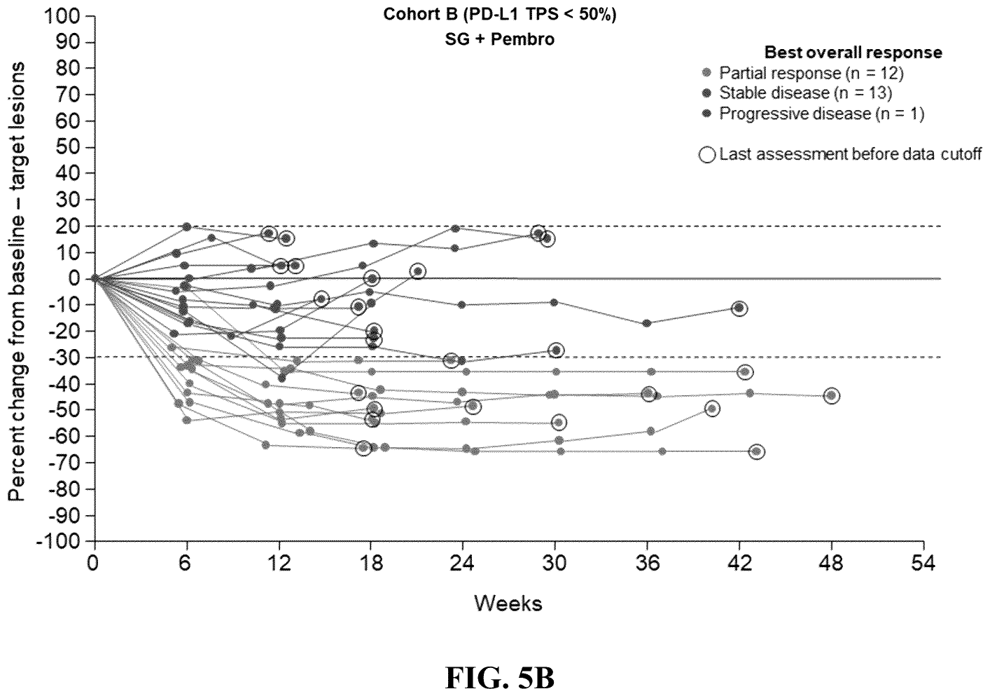

In certain embodiments, hRS7 is described, e.g., in WO2003074566, FIGS. 3 and 4, incorporated by reference in its entirety.

In certain embodiments, SG is represented by Formula I as shown below.

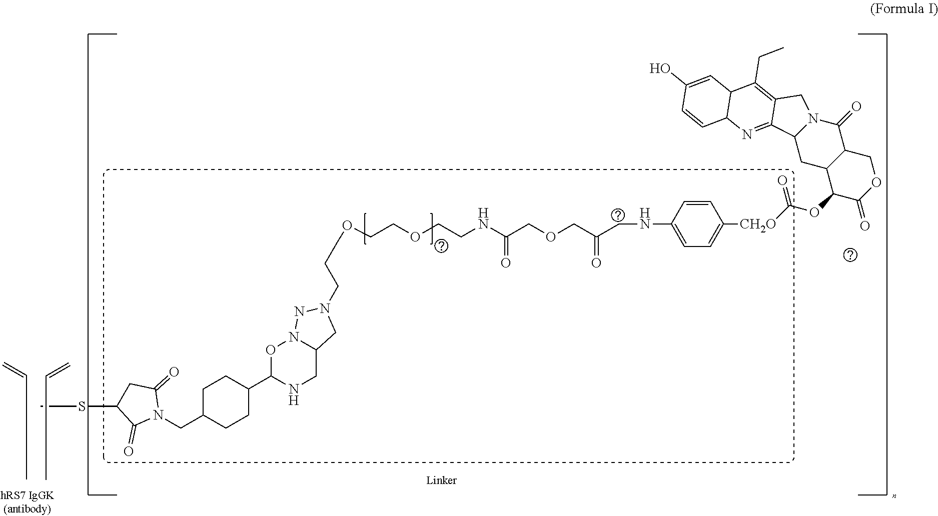

In certain embodiments, ADC comprises drug molecules linked to the antibody moieties in various stoichiometric molar ratios depending on the configuration of the antibody and, at least in part, the method used to effect configuration. In certain embodiments, the drug-antibody ratio (“DAR”) is about 7.6. In certain embodiments, the DAR is about 7.0 to about 8.0.

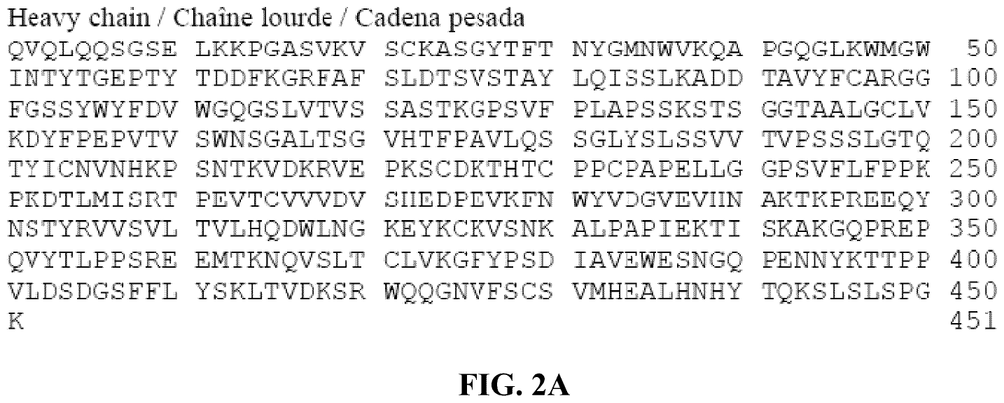

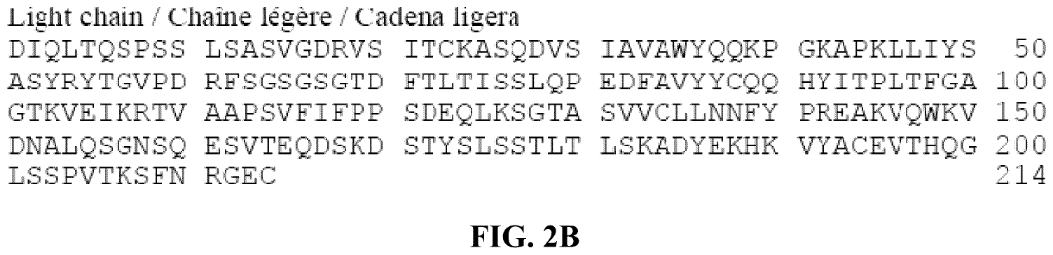

In certain embodiments, the hRS7 antibody in SG comprises the heavy chain as shown in SEQ ID NO.: 1 and light chain as shown in SEQ ID NO.: 2 (as shown in Table 1 and in FIGS. 2A-2B). In certain embodiments, the hRS7 antibody in SG comprises two heavy chains each having the sequence as shown in SEQ ID NO.: 1, and two light chains each having the sequence as shown in SEQ ID NO.: 2.

TABLE 1
Amino Acid Sequences of hRS7 antibody

| SEQ | | |
|---|---|---|
| ID NO | Description | Amino Acid Sequence |
| 1 | hRS7 heavy | QVQLQQSGSELKKPGASVKVSCKASGYTFTNYGMNWVKQAPGQGLK |
| | chain | WMGWINTYTGEPTYTDDFKGRFAFSLDTSVSTAYLQISSLKADDTA |
| | | VYFCARGGFGSSYWYFDVWGQGSLVTVSSASTKGPSVFPLAPSSKS |
| | | TSGGTAALGCLVKDYFPEPVTVSWNSGALTSGVHTFPAVLQSSGLY |
| | | SLSSVVTVPSSSLGTQTYICNVNHKPSNTKVDKRVEPKSCDKTHTC |
| | | PPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVS |
| | | LTCLVKGFYPSDIAVEWESNGQPENNYKTTPPVLDSDGSFFLYSKL |
| | | TVDKSRWQQGNVFSCSVMHEALHNHYTQKSLSLSPGK |
| 2 | hRS7 light | DIQLTQSPSSLSASVGDRVSITCKASQDVSIAVAWYQQKPGKAPKL |
| | chain | LIYSASYRYTGVPDRFSGSGSGTDFTLTISSLQPEDFAVYYCQQHY |
| | | ITPLTFGAGTKVEIKRTVAAPSVFIFPPSDEQLKSGTASVVCLLNN |
| | | FYPREAKVQWKVDNALQSGNSQESVTEQDSKDSTYSLSSTLTLSKA |
| | | DYEKHKVYACEVTHQGLSSPVTKSENRGEC |
| 13 | HCDR1 | NYGMN |
| 14 | HCDR2 | WINTYTGEPTYTDDFKG |
| 15 | HCDR3 | GGFGSSYWYFDV |
| 16 | LCDR1 | KASQDVSIAVA |
| 17 | LCDR2 | SASYRYT |
| 18 | LCDR3 | QQHYITPLT |
| 19 | VH | QVQLQQSGSELKKPGASVKVSCKASGYTFTNYGMNWVKQAPGQGLK |
| | | WMGWINTYTGEPTYTDDFKGRFAFSLDTSVSTAYLQISSLKADDTA |
| | | VYFCARGGFGSSYWYFDVWGQGSLVTVSS |
| 20 | VL | DIQLTQSPSSLSASVGDRVSITCKASQDVSIAVAWYQQKPGKAPKL |
| | | LIYSASYRYTGVPDRFSGSGSGTDFTLTISSLQPEDFAVYYCQQHY |
| | | ITPLTFGAGTKVEIKR |

Exemplary anti-Trop-2 ADCs that can be used in the methods provided herein are described, for example, in U.S. Pat. Nos. 7,999,083 and 9,028,833, which are hereby incorporated herein by reference in their entireties.

### Anti-PD-1 Antibody or Antigen Binding Fragment Thereof

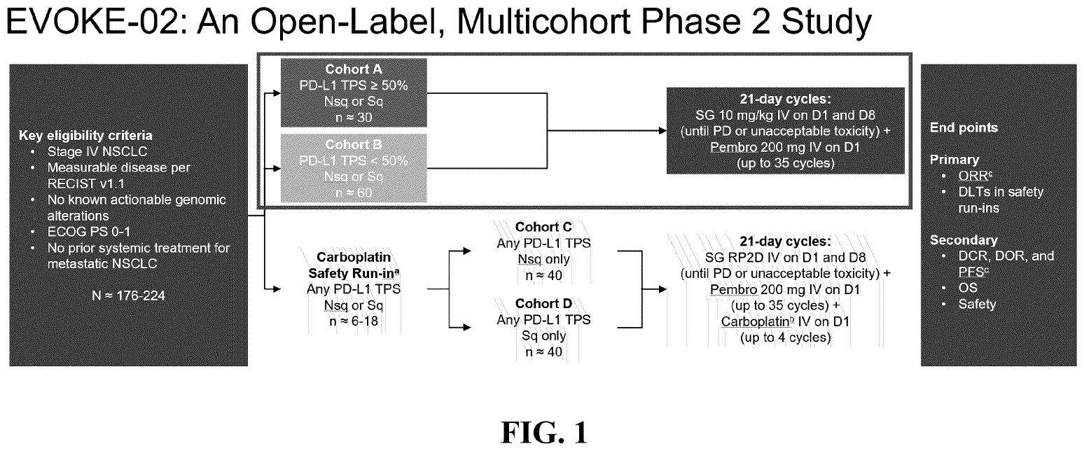

Examples of monoclonal antibodies (mAbs) that bind to human PD-1, useful in the treatment methods and uses of the invention, are described in U.S. Pat. Nos. 7,521,051, 8,008,449, and 8,354,509. Specific anti-human PD-1 mAbs useful as a PD-1 antagonist in the treatment methods of the present disclosure include: pembrolizumab (formerly known as MK-3475, SCH 900475 and lambrolizumab), a humanized IgG4 mAb with the structure described in WHO) Drug Information, Vol. 27, No. 2, pages 161-162 (2013) and which comprises the heavy and light chain amino acid sequences shown in FIG. 1, and the humanized antibodies h409A11, h409A16 and h409A17, which are described in WO 2008/156712 and in Table 2.

In some embodiments of the methods of the disclosure, the anti-PD-1 antibody, or antigen binding fragment thereof, comprises: (a) light chain CDRs comprising a sequence of amino acids as set forth in SEQ ID NOs: 3, 4 and 5, and (b) heavy chain CDRs comprising a sequence of amino acids as set forth in SEQ ID NOs: 8, 9 and 10.

In some embodiments, the anti-PD-1 antibody or antigen binding fragment thereof is a human antibody. In other embodiments, the anti-PD-1 antibody or antigen binding fragment thereof is a humanized antibody. In other embodiments, the anti-PD-1 antibody or antigen binding fragment thereof is a chimeric antibody. In specific embodiments, the anti-PD-1 antibody or antigen binding fragment thereof is a monoclonal antibody.

In other embodiments of the treatment methods of the present disclosure, the anti-PD-1 antibody, or antigen binding fragment thereof, specifically binds to human PD-1 and comprises (a) a heavy chain variable region comprising an amino acid sequence as set forth in SEQ ID NO:11, or a variant thereof, and (b) a light chain variable region comprising an amino acid sequence selected from the group consisting of SEQ ID NO:6.

A variant of a heavy chain variable region sequence or full-length heavy chain sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence or full-length light chain sequence is identical to the reference sequence except having up to five conservative amino acid substitutions in the framework region (i.e., outside of the CDRs), and preferably has less than four, three or two conservative amino acid substitution in the framework region.

In another embodiment of the treatment methods of the present disclosure, the anti-PD-1 antibody or antigen-binding fragment thereof is a monoclonal antibody which specifically binds to human PD-1 and comprises (a) a heavy chain comprising or consisting of a sequence of amino acids as set forth in SEQ ID NO:12, or a variant thereof; and (b) a light chain comprising or consisting of a sequence of amino acids as set forth in SEQ ID NO:7, or a variant thereof.

In yet another embodiment of the treatment methods of the present disclosure, the anti-PD-1 antibody or antigen-binding fragment thereof is a monoclonal antibody which specifically binds to human PD-1 and comprises (a) a heavy chain comprising or consisting of a sequence of amino acids as set forth in SEQ ID NO:12 and (b) a light chain comprising or consisting of a sequence of amino acids as set forth in SEQ ID NO:7.

TABLE 2
Exemplary PD-1 Antibody Sequences

| | | |
|---|---|---|
| Antibody | | SEQ |
| Feature | Amino Acid Sequence | ID NO. |

Pembrolizumab Light Chain

| | | |
|---|---|---|
| CDR1 | RASKGVSTSGYSYLH | 3 |
| CDR2 | LASYLES | 4 |
| CDR3 | QHSRDLPLT | 5 |
| Variable | EIVLTQSPATLSLSPGERATLSCRASKGVSTSGYSYLHWY | 6 |
| Region | QQKPGQAPRLLIYLASYLESGVPARFSGSGSGTDFTLTISSL | |
| | EPEDFAVYYCQHSRDLPLTFGGGTKVEIK | |
| Light Chain | EIVLTQSPATLSLSPGERATLSCRASKGVSTSGYSYLHWY | 7 |
| | QQKPGQAPRLLIYLASYLESGVPARFSGSGSGTDFTLTISSL | |
| | EPEDFAVYYCQHSRDLPLTFGGGTKVEIKRTVAAPSVFIFP | |
| | PSDEQLKSGTASVVCLLNNFYPREAKVQWKVDNALQSGN | |
| | SQESVTEQDSKDSTYSLSSTLTLSKADYEKHKVYACEVTH | |
| | QGLSSPVTKSFNRGEC | |

Pembrolizumab Heavy Chain

| | | |
|---|---|---|
| CDR1 | NYYMY | 8 |
| CDR2 | GINPSNGGTNFNEKFKN | 9 |
| CDR3 | RDYRFDMGFDY | 10 |
| Variable | QVQLVQSGVEVKKPGASVKVSCKASGYTFTNYYMYWVR | 11 |
| Region | QAPGQGLEWMGGINPSNGGTNFNEKFKNRVTLTTDSSTT | |
| | TAYMELKSLQFDDTAVYYCARRDYRFDMGFDYWGQGTT | |
| | VTVSS | |
| Heavy | QVQLVQSGVEVKKPGASVKVSCKASGYTFTNYYMYWVR | 12 |
| Chain | QAPGQGLEWMGGINPSNGGTNFNEKFKNRVTLTTDSSTT | |
| | TAYMELKSLQFDDTAVYYCARRDYRFDMGFDYWGQGTT | |
| | VTVSSASTKGPSVFPLAPCSRSTSESTAALGCLVKDYFPEP | |
| | VTVSWNSGALTSGVHTFPAVLQSSGLYSLSSVVTVPSSSL | |
| | GTKTYTCNVDHKPSNTKVDKRVESKYGPPCPPCPAPEFLG | |
| | GPSVFLFPPKPKDTLMISRTPEVTCVVVDVSQEDPEVQFN | |
| | WYVDGVEVHNAKTKPREEQFNSTYRVVSVLTVLHQDWL | |
| | NGKEYKCKVSNKGLPSSIEKTISKAKGQPREPQVYTLPPSQ | |
| | EEMTKNQVSLTCLVKGFYPSDIAVEWESNGQPENNYKTT | |
| | PPVLDSDGSFFLYSRLTVDKSRWQEGNVFSCSVMHEALH | |
| | NHYTQKSLSLSLGK | |

In one embodiment, the anti-PD-1 antibody or antigen-binding fragment thereof comprises a heavy chain constant region, e.g., a human constant region, such as g1, g2, g3, or g4 human heavy chain constant region or a variant thereof. In another embodiment, the anti-PD-1 antibody or antigen-binding fragment thereof comprises a light chain constant region, e.g., a human light chain constant region, such as lambda or kappa human light chain region or a variant thereof. By way of example, and not limitation, the human heavy chain constant region can be g4 and the human light chain constant region can be kappa. In an alternative embodiment, the Fc region of the antibody is g4 with a Ser228Pro mutation (Schuurman, J et. al., Mol. Immunol. 38:1-8, 2001). In some embodiments, different constant domains may be appended to humanized VL and VH regions derived from the CDRs provided herein. For example, if a particular intended use of an antibody (or fragment) of the present invention were to call for altered effector functions, a heavy chain constant domain other than human IgG1 may be used, or hybrid IgG1/IgG4 may be utilized. Although human IgG1 antibodies provide for long half-life and for effector functions, such as complement activation and antibody-dependent cellular cytotoxicity, such activities may not be desirable for all uses of the antibody. In such instances a human IgG4 constant domain, for example, may be used. The present invention includes the use of anti-PD-1 antibodies or antigen-binding fragments thereof which comprise an IgG4 constant domain. In one embodiment, the IgG4 constant domain can differ from the native human IgG4 constant domain (Swiss-Prot Accession No. P01861.1) at a position corresponding to position 228 in the EU system and position 241 in the KABAT system, where the native Ser108 is replaced with Pro, in order to prevent a potential inter-chain disulfide bond between Cys106 and Cys109 (corresponding to positions Cys 226 and Cys 229 in the EU system and positions Cys 239 and Cys 242 in the KABAT system) that could interfere with proper intra-chain disulfide bond formation. See Angal et al. (1993) Mol. Imunol. 30:105. In other instances, a modified IgG1 constant domain which has been modified to increase half-life or reduce effector function can be used.

In another embodiment, the anti-PD-1 antibody or antigen binding fragment thereof has a variable light domain and/or a variable heavy domain with at least 95%, 90%, 85%, 80%, 75% or 50% sequence identity to one of the variable light domains or variable heavy domains described above, and exhibits specific binding to PD-1. In another embodiment of the methods of treatment of the invention, the anti-PD-1 antibody or antigen binding fragment thereof comprises variable light and variable heavy domains having up to 1, 2, 3, 4, or 5 or more amino acid substitutions, and exhibits specific binding to PD-1.

### Definitions

The singular forms “a,” “an,” and “the” include the plural referents unless the context dictates otherwise.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error, such as, for example, within 15%, 10%, or 5%.

“Actionable genomic alteration(s),” is known in the art, i.e., a gene is actionable if it has an established biologic role in cancer and there is a clinically available drug to which the gene confers sensitivity or resistance, where actionability can be applicable to all or select alteration or tumor types.

“Advanced disease” refers to stage III disease which has not spread or cancers that are unlikely to be cured or controlled long-term with treatment, as they have spread to distant locations

“Anti-cancer agent” refers to an approved or investigational agent(s) for use, or that are being evaluated in, cancer. It can refer to systemic and targeted therapy.

“Metastatic cancer or disease” is a sub-category of advanced disease. It refers to Stage IV disease and/or cancers that are unlikely to be cured or controlled long-term with treatment, as they have spread to distant locations.

“Locally advanced disease,” as used herein, refers to a disease state in which cancer cells have begun to spread out from the initial site of origin but have not yet spread to other parts of the body.

“Antibody-drug conjugate” or “ADC” generally refers to a compound comprising an antibody targeting a tumor antigen and an anticancer agent drug or payload, optionally connected by a linker. In certain embodiments, the ADC is SG. In certain embodiments, the ADC is an ADC that is approved for NSCLC, or in the clinic being evaluated for NSCLC. In certain embodiments, the ADC comprises a Trop2-targeting antibody. In certain embodiments, “ADC” refers to the compound of Formula I, which has an anti-Trop-2 antibody (sacituzumab) linked to the drug or payload SN38.

An “antibody fragment” or “antigen binding fragment” refers to a molecule other than an intact antibody that comprises a portion of an intact antibody that binds the antigen to which the intact antibody binds. The antibody fragment retains the ability to bind specifically to the antigen bound by the full-length antibody, e.g., fragments that retain one or more CDR regions, e.g. all six CDRs. Examples of antibody fragments include but are not limited to Fv, Fab, Fab′, Fab′-SH, F (ab′) 2; diabodies; linear antibodies; single-chain antibody molecules (e.g., scFv); and multi-specific antibodies formed from antibody fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g., without producing undesired results such as false positives. Antibodies, or binding fragments thereof, useful in the present invention will bind to the target protein with an affinity that is at least two-fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g., the amino acid sequence of a mature human PD-1 or human PD-L1 molecule, if it binds to polypeptides comprising that sequence but does not bind to proteins lacking that sequence.

“Baseline” as used herein, is the time within approximately 28 days prior to initiation of therapy, i.e., within approximately 28 days prior to day 1 of cycle 1. In certain embodiments, baseline refers to approximately day −28 to day −1 prior to day 1 of cycle 1. In certain embodiments, the baseline is the time within approximately 28 days+/−7 days prior to the initiation of therapy. In certain embodiments, baseline is the time from one measurement to another, i.e., baseline can refer to an initial time point when a measurement, i.e., a measurement of a lesion, is compared between two time points. The initial time point, in this instance, does not necessarily correspond with the initiation of treatment.

“Blinded Independent Central Review” (“BICR”) refers to the process by which radiologic exams and selected clinical data, performed as part of a clinical trial protocol, are submitted to a central location for independent review.

“Chemotherapy-naïve,” as used herein, refers to not having received chemotherapy, i.e., not having received chemotherapy for advanced or metastatic NSCLC.

“Co-administering,” refers to the administration of two or more drugs or therapies to a patient. As used herein, “co-administration” refers to administration of two or more drugs during the same time period. In certain embodiments, “co-administration” refers to administration of two or more drugs during the same dosing cycle. For example, if the dosing cycle is 21-days, then “co-administration” can refer to administration of drug 1 on day 1, and drug 2 on day 1 or any other day within the 21-day dosing cycle. In certain embodiments, “co-administration” refers to 2 two or more drugs that are administered together within at least one dosing cycle. The co-administration of the two therapies can also be via the same or different routes of administration.

“Clinical efficacy” or “clinical activity” refers to clinical efficacy or activity in human patient(s). In certain embodiments, clinical activity or clinical efficacy refers to a partial or complete response. In certain embodiments, clinical efficacy or clinical activity, when used in the context of a population of patients, comprises ORR, DOR, and/or DCR. In certain embodiments, improvement of clinical efficacy or clinical activity, when used in the context of a comparison between two or more agents, can be used to describe a clinically meaningful benefit to the human patient of one agent compared to the other. As used herein “clinically meaningful benefit” (or grammatical variations thereof) refers to results/findings that improve medical care resulting in improvement in the individual's physical function, his/her mental status, and/or ability to engage in social life. The term improvement of quality of life in medical care deals with both subjective and objective terms. Objective terms may be duration of remission of disease, etc. Subjective terms may be improvement in the quality of life.

“Complete Response” (“CR”) refers to disappearance of all target lesions.

“CDR” or “CDRs” as used herein means complementarity determining region(s) in an immunoglobulin variable region, defined using the Kabat numbering system, unless otherwise indicated.

“Concomitant administration” or “concurrent administration,” can be used interchangeably, and refer to administration of two or more drugs or therapies during the same dosing cycle. Each of the drugs can be approved or investigational for the indication-of-interest.

“Disease Control Rate” (“DCR”) is defined as the proportion of patients who achieve a CR, PR or SD (stable disease) as assessed by IRC per RECIST Version 1.1 and/or by investigator assessment

“Duration of response” (“DOR”) refers to the time (e.g., months) from onset of response to progression or death.

“First line treatment” as is known in the art refers to the initial therapy for a particular disease.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352:624-628 and Marks et al. (1991) J. Mol. Biol. 222:581-597, for example. See also Presta (2005) J. Allergy Clin. Immunol. 116:731.

“NSCLC histology” as used herein, refers to whether the NSCLC is squamous or non-squamous.

“Objective Responsive Rate” (“ORR”) refers to the proportion of subjects who have measurable disease at baseline and achieve a complete response (CR) or partial response (PR) that is confirmed at least 4 weeks later. In certain embodiments, ORR is assessed by an independent review committee (IRC) according to the Response Evaluation Criteria in Solid Tumors (RECIST) Version 1.1. In certain embodiments, ORR is assessed by the investigator. In certain embodiments, the primary analysis of ORR, as assessed per RECIST Version 1.1, is performed by estimating the proportion of patients who achieve a confirmed CR or PR in the FAS for each cohort respectively, and is 2-sided confidence interval (“CI”) with the Clopper-Pearson exact method.

“Partial Response” (“PR”), refers to a >30% decrease in the sum of the longest diameter (“LD”) of target lesions, taking as reference the baseline sum LD. In certain embodiments, lesions are measured by computed tomography or MRI scans with IV contrast (unless contrast use is medically contraindicated). Lesions in the chest, abdomen, pelvis, and any other involved disease sites are measured in all patients according to the study procedures (as shown in FIG. 3) at baseline and every 6 weeks after initiation of study treatment for the first 12 months and then every 9 weeks until initiation of subsequent anticancer therapy or termination of study by sponsor, whichever occurs first. For patients with evidence of CR or PR, a confirmatory scan is performed at least 4 weeks after initial documentation of response. In certain embodiments, tumor response and progression are determined using Response Evaluation Criteria in Solid Tumors (RECIST) Version 1.1 by IRC. In certain embodiments, PR refers to a 50% or greater size in the measurable tumor(s).

“Patient” or “subject,” as used herein, refer to a human having a particular disease. In certain embodiments, the patient is an adult patient. In certain embodiments, the patient is a pediatric patient.

“Pembrolizumab” (formerly known as MK-3475, SCH 900475, and lambrolizumab), alternatively referred to herein as “pembro,” is a humanized IgG4 mAb with the structure described in WHO Drug Information, Vol. 27, No. 2, pages 161-162 (2013) and which comprises the heavy and light chain amino acid sequences and CDRs described in Table 2. Pembrolizumab has been approved by the U.S. FDA as described in the Prescribing Information for KEYTRUDA™ (Merck & Co., Inc., Rahway, NJ, USA; initial U.S. approval 2014, updated March 2021).

As used herein, a “pembrolizumab variant” or “a variant thereof” pertaining to a pembrolizumab sequence means a monoclonal antibody that comprises heavy chain and light chain sequences that are substantially identical to those in pembrolizumab, except for having three, two or one conservative amino acid substitutions at positions that are located outside of the light chain CDRs and six, five, four, three, two or one conservative amino acid substitutions that are located outside of the heavy chain CDRs, e.g., the variant positions are located in the FR regions or the constant region, and optionally has a deletion of the C-terminal lysine residue of the heavy chain. In other words, pembrolizumab and a pembrolizumab variant comprise identical CDR sequences, but differ from each other due to having a conservative amino acid substitution at no more than three or six other positions in their full-length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

“Progression Free Survival” (“PFS”) refers to the time from first dose until the date of the of disease progression or death (whichever occurs first) according to ICR using RECIST 1.1.

“Refractory” is used to describe when the disease does not respond to treatment or when the response to treatment does not last very long.

“Relapsed” refers to a disease that reappears or grows again after a period of remission.

“Standard of care,” as used herein refers to a preferred treatment regimen for a particular disease or indication, such as a treatment that a governmental regulatory agency has approved for the particular disease or indication.

“Targeted therapie(s)” refers to small molecule inhibitors, monoclonal antibodies, tumor agnostic treatments, and the like, in cancer treatment.

“Therapeutically effective amount” or “effective amount” is defined as an amount of compound/drug to treat the disease or disorders and achieve clinical efficacy.

“Treatment” (or grammatical variations thereof), refers to medical care that results in clinically meaningful benefit or clinically meaningful improvement in human patient(s) or human subject(s).

“Treatment emergent adverse effects” (“TEAEs”), as used herein, refers to adverse effects reported in patients who have received at least one dose of a treatment.

“Treatment-naïve,” as used herein, refers to not having received treatment, i.e., not having received treatment for advanced or metastatic NSCLC.

“Tumor proportion score” (“TPS”), i.e., PD-L1 TPS, refers to PD-L1 protein expression determined using the percentage of viable tumor cells showing partial or complete membrane staining at any intensity, as is known in the art. In certain embodiments, FDA-validated tests may be used to measure TPS. In certain embodiments, TPS refers to PD (L)-1 protein expression.

### Recommended Dosing Regimen

In certain embodiments, SG is administered as an intravenous infusion at a dosage of 10 mg/kg once weekly on days 1 and 8 of continuous 21-day treatment cycles. In certain embodiments, SG is administered until disease progression or unacceptable toxicity.

In some embodiments, the anti-PD-1 antibody or antigen-binding fragment thereof (e.g., pembrolizumab) is administered to a subject in need thereof about once every two weeks, about once every three weeks, or about once every four weeks. In some embodiments, the anti-PD-1 antibody or antigen-binding fragment thereof is administered to a subject in need thereof about once every two weeks, about once every three weeks, about once every four weeks, or about once every six weeks. In some embodiments, the anti-PD-1 antibody or antigen-binding fragment thereof is administered to a subject in need thereof once every two weeks. In some embodiments, the anti-PD-1 antibody or antigen-binding fragment thereof is administered to a subject in need thereof once every three weeks. In some embodiments, the anti-PD-1 antibody or antigen-binding fragment thereof is administered to a subject in need thereof once every six weeks. In some embodiments, the anti-PD-1 antibody or antigen-binding fragment thereof is administered to a subject in need thereof once every four weeks. In some embodiments, the anti-PD-1 antibody or antigen-binding fragment thereof is administered about once every 14, 15, 16, 17, 18, 19, 20, or 21 days.

In some embodiments, the anti-PD-1 antibody or antigen-binding fragment thereof (e.g., pembrolizumab) is administered at a dose of about 200 mg every 3 weeks, at a dose of about 400 mg every 6 weeks, or at a dose of about 2 mg/kg every 3 weeks (up to a maximum of 200 mg).

In some embodiment, the anti-PD-1 antibody or antigen-binding fragment thereof (e.g., pembrolizumab) is administered at a dose of about 2 mg/kg. In some embodiment, pembrolizumab is administered at a dose of about 2 mg/kg every three weeks. In particular embodiments, the patient is a pediatric patient.

In some embodiment, the anti-PD-1 antibody or antigen-binding fragment thereof (e.g., pembrolizumab) is administered as a 30 minute (−5 minutes/+10 minutes) intravenous infusion. In one embodiment, the selected dose of the anti-PD-1 antibody or antigen-binding fragment thereof is administered by IV infusion over a time period of between 25 and 40 minutes, or about 30 minutes.

In one aspect, the anti-PD-1 antibody or antigen-binding fragment thereof (e.g., pembrolizumab) in included in a pharmaceutical composition with a pharmaceutically acceptable carrier or diluent and may include additional pharmaceutically acceptable excipients.

### Co-administration Dosing Regimen

In certain embodiments, SG and pembrolizumab are co-administered. In certain embodiments, 10 mg/kg SG is administered as an intravenous infusion on day 1 and day 8 of one or more 21-day treatment cycles, and 200 mg pembrolizumab is administered as an intravenous infusion on the day 1 of the one or more 21-day treatment cycles. In certain embodiments, treatment with the combination of SG and pembrolizumab is initiated on the same day of the 21-day dosing cycle, i.e., day 1 of the 21-day dosing cycle. In certain embodiments, on treatment days when both SG and pembrolizumab are administered, i.e., day 1 of the 21-day treatment cycle, pembrolizumab is administered first, followed by SG. In certain embodiments, on treatment days when both SG and pembrolizumab are administered, i.e., day 1 of the 21-day treatment cycle, SG is administered first, followed by pembrolizumab. In certain embodiments, pembrolizumab is administered for no more than (35) 21-day dosing cycles. In certain embodiments, SG is administered until disease progression or unacceptable toxicity. In certain embodiments, SG is administered in one or more 21-day dosing cycles prior to co-administration of SG and pembrolizumab. In certain embodiments, pembrolizumab is not administered in one or more 21-day dosing cycles prior to co-administration of SG and pembrolizumab.

### Dose Modifications for Adverse Reactions

In certain embodiments, the dosage of 10 mg/kg of SG is withheld, modified, or discontinued to manage adverse reactions as described in Tables 3 and 4. In certain embodiments, the dosage of SG is not re-escalated after a dose reduction for an adverse reaction has been made.

TABLE 3
Dose Modifications for Adverse Reactions (Severe Neutropenia)

| | | |
|---|---|---|
| Adverse Reaction | Occurrence | Dose Modification |

Severe Neutropenia [see Warnings and Precautions (5.1)]

| | | |
|---|---|---|
| Grade 4 neutropenia ≥7 days | First | 25% dose reduction and |
| OR | | administer granulocyte-colony |
| Grade 3-4 febrile neutropenia, | | stimulating factor (G-CSF) |
| OR | Second | 50% dose reduction and |
| At time of scheduled treatment, Grade 3-4 | | administer G-CSF |
| neutropenia which delays dosing by 2 or 3 weeks | Third | Discontinue treatment and |
| for recovery to ≤ Grade 1 | | administer G-CSF |
| At time of scheduled treatment, Grade 3-4 | First | Discontinue treatment and |
| neutropenia which delays dosing beyond 3 weeks | | administer G-CSF |
| for recovery to ≤ Grade 1 | | |

TABLE 4
Dose Modifications for Adverse Reactions (Severe Non-Neutropenic Toxicity)

| | | |
|---|---|---|
| Adverse Reaction | Occurrence | Dose Modification |

Severe Non-Neutropenic Toxicity

| | | |
|---|---|---|
| Grade 4 non-hematologic toxicity of any duration | First | 25% dose reduction |
| OR | | |
| Any Grade 3-4 nausea, vomiting or diarrhea due to | Second | 50% dose reduction |
| treatment that is not controlled with antigmetics and anti- | | |
| diarrheal agents [see Warnings and Precautions | Third | Discontinue treatment |
| (5.2, 5.4)] | | |
| OR | | |
| Other Grade3-4 non-hematologic toxicity persisting >48 | | |
| hours despite optimal medical management, | | |
| OR | | |
| At time of scheduled treatment, Grade 3-4 non- | | |
| neutropenic hematologic or non-hematologic toxicity, | | |
| which delays dose by 2 or 3 weeks for recovery to ≤ | | |
| Grade 1 | | |
| In the event of Grade 3-4 non-neutropenic hematologic or | First | Discontinue treatment |
| non-hematologic toxicity, which does not recover to ≤ | | |
| Grade 1 within 3 weeks | | |

### Patient Populations

In certain embodiments, patients treated with the methods disclosed herein have NSCLC. In certain embodiments, the NSCLC is advanced. In certain embodiments, the NSCLC is advanced or metastatic. In certain embodiments, the NSCLC is metastatic. In certain embodiments, the NSCLC is treatment naïve. In certain embodiments, the NSCLC is chemotherapy naïve.

In certain embodiments, the methods disclosed herein are used to treat first line NSCLC. In certain embodiments, the methods disclosed herein are used to treat first line advanced or metastatic NSCLC. In certain embodiments, the methods disclosed herein are used to treat first line metastatic NSCLC.

In certain embodiments, patients treated with the methods disclosed herein have treatment naïve NSCLC. In certain embodiments, patients treated with the methods disclosed herein have chemotherapy naive NSCLC. In certain embodiments, patients treated with the methods disclosed herein have not had prior therapy for NSCLC. In certain embodiments, patients treated with the methods disclosed herein have not had prior systemic therapy for NSCLC. In certain embodiments, the patients have not had prior systemic therapy for metastatic NSCLC. In certain embodiments, the patients have received adjuvant or neoadjuvant therapy that was completed at least 6 months prior to development of metastatic disease. In certain embodiments, the patients have not received adjuvant or neoadjuvant therapy that was completed at least 6 months prior to development of metastatic disease. In certain embodiments, the patient having advanced or metastatic NSCLC has not had prior therapy for advanced or metastatic NSCLC prior to co-administration of SG and pembrolizumab as disclosed herein. In certain embodiments, the patient has not had prior targeted therapy (either approved or investigational) for NSCLC. In certain embodiments, the patient has not had prior systemic therapy for NSCLC. In certain embodiments, the patient has not had prior systemic therapy for metastatic NSCLC.

In certain embodiments, patients treated with the methods disclosed herein do not have genomic alterations in EGFR. In certain embodiments, the patients do not have genomic alterations in ALK. In certain embodiments, the patients do not have actionable genomic alterations in EFGR. In certain embodiments, the patients do not have actionable genomic alterations in ALK. In certain embodiments, the patients do not have actionable genomic alterations in EGFR and ALK. In certain embodiments, if patient status is not known, testing is required. In certain embodiments, if patient status is not known, testing is not required.

In certain embodiments, patients treated with the methods disclosed herein do not have known genomic alterations in ROS1, NTRK, BRAF, RET and/or other actionable driver oncogenes with approved therapies for frontline treatment. In certain embodiments, if patient status is not known, testing is not required.

In certain embodiments, patients treated with the methods disclosed herein do not have a mixed small cell lung cancer (“SCLC”)/NSCLC histology.

In certain embodiments, patients treated with the methods disclosed herein have squamous NSCLC. In certain embodiments, patients treated with the methods disclosed herein have non-squamous NSCLC. In certain embodiments, patients treated with the methods disclosed herein have both squamous and non-squamous NSCLC.

In certain embodiments, the methods disclosed herein do not comprise administering an anti-TIGIT antibody to the patient. In certain embodiments, the patient does not receive an anti-TIGIT antibody during the one or more 21-day treatment cycles. In certain embodiments, the method does not comprise concomitant administration of an anti-TGIT antibody.

In certain embodiments, the methods disclosed herein do not comprise concomitant administration of an anti-cancer agent. In certain embodiments, the methods disclosed herein do not comprise concomitant administration of chemotherapy. In certain embodiments, the methods disclosed herein do not comprise concomitant administration of a platinum-based chemotherapy. In certain embodiments, the methods disclosed herein do not comprise concomitant administration of a pemetrexed. In certain embodiments, the methods disclosed herein do not comprise concomitant administration of pemetrexed and platinum-based chemotherapy. In certain embodiments, the methods disclosed herein do not comprise concomitant administration of carboplatin. In certain embodiments, the methods disclosed herein do not comprise concomitant administration of paclitaxel/paclitaxel protein-bound. In certain embodiments, the methods disclosed herein do not comprise concomitant administration of carboplatin and paclitaxel/paclitaxel protein-bound. In certain embodiments, the methods disclosed herein do not comprise concomitant administration of pemetrexed, platinum-based chemotherapy, carboplatin, and paclitaxel/paclitaxel protein-bound.

In certain embodiments, the patients treated with the methods disclosed herein have not had platinum-based chemotherapy for NSCLC or metastatic NSCLC. In certain embodiments, the patients treated with the methods disclosed herein have not had immune-checkpoint inhibitor therapy for NSCLC or metastatic NSCLC. In certain embodiments, the patients treated with the methods disclosed herein have not had prior docetaxel therapy for NSCLC. In certain embodiments, the patients treated with the methods disclosed herein have not had prior docetaxel therapy for metastatic NSCLC.

In certain embodiments, the method further includes administering one or more platinum-based chemotherapy. In some embodiments, the patient receives platinum-based chemotherapy during one or more 21-day treatment cycles. In some embodiments, the platinum-based chemotherapy is administered at day 1 of one or more 21-day treatment cycles. In some embodiments, the platinum-based chemotherapy is continued for up to 4 cycles.

Any platinum-based chemotherapy known can be used in the method. In some embodiments, the platinum-based chemotherapy is selected from a list consisting of cisplatin, carboplatin, oxaliplatin, and platinum nanocluster-based (Pt NC-based) nanodrugs.

Cisplatin is the first generation of Pt-based anti-cancer drugs, discovered in the late 1960s and approved for cancer treatment in 1978. Cisplatin has a therapeutic effect on many malignant tumors, such as breast, ovarian, and colorectal cancers. The second-generation platinum chemotherapy drug carboplatin was developed based on cisplatin. Compared with cisplatin, carboplatin exhibits a lower hydration rate due to the bidentate cyclobutane dicarboxylic acid ligands and has high biosafety with greatly reduced systemic toxicity, including hepatotoxicity, nephrotoxicity, neurotoxicity, and ototoxicity. Because of its lower toxicity, carboplatin can be used as high-dose chemotherapy for aggressive tumors. Oxaliplatin is the third generation of platinum clinical drug. The mechanism of action of oxaliplatin is similar to cisplatin, without producing cross-resistance with cisplatin or carboplatin. Therefore, oxaliplatin and cisplatin can achieve a complementary effect in clinical anti-cancer treatment and have been widely used (Zhang et al., Theranostics, 12 (5): 2115-2132 (2022)).

In some embodiments, the platinum-based chemotherapy is cisplatin. In some embodiments, cisplatin is administered at a dosage of 75 mg/m2. In other embodiments, the platinum-based chemotherapy is carboplatin. In some embodiments, carboplatin is administered at a dosage of AUC5. In some embodiments, the cisplatin or cisplatin treatment is continued for up to 4 cycles. A person skilled in the art can adjust the dosage and cycle of the platinum-based chemotherapy based on the conditions of the subject.

### Efficacy

In certain embodiments, treatment of NSCLC in a population of patients each having NSCLC with the methods disclosed herein achieves an ORR of about 44%, about 54%, or about 75%. In certain embodiments, treatment of NSCLC in a population of patients each having NSCLC with the methods disclosed herein achieves an ORR of 44%, 54%, or 75%. In certain embodiments, treatment of NSCLC in a population of patients each having NSCLC with the methods disclosed herein achieves an ORR of at least 44%. In certain embodiments, treatment of NSCLC in a population of patients each having NSCLC with the methods disclosed herein achieves an ORR of at least 54%. In certain embodiments, treatment of NSCLC in a population of patients each having NSCLC with the methods disclosed herein achieves an ORR of at least 75%. In certain embodiments, treatment of NSCLC in a population of patients each having NSCLC with the methods discloses herein achieves an ORR of about 44%. In certain embodiments, treatment of NSCLC in a population of patients each having NSCLC with the methods discloses herein achieves an ORR of 44%. In certain embodiments, treatment of NSCLC in a population of patients each having NSCLC with the methods disclosed herein achieves an ORR of about 54%. In certain embodiments, treatment of NSCLC in a population of patients each having NSCLC with the methods discloses herein achieves an ORR of 54%. In certain embodiments, treatment of a NSCLC in a population of patients each having NSCLC achieves an ORR of about 75%. In certain embodiments, treatment of NSCLC in a population of patients each having NSCLC with the methods discloses herein achieves an ORR of 75%.

In certain embodiments, treatment of NSCLC in a population of patients each having NSCLC with the methods disclosed herein achieves an ORR of about 44%, about 56%, or about 69%. In certain embodiments, treatment of NSCLC in a population of patients each having NSCLC with the methods disclosed herein achieves an ORR of 44%, 56%, or 69%. In certain embodiments, treatment of NSCLC in a population of patients each having NSCLC with the methods disclosed herein achieves an ORR of at least 44%. In certain embodiments, treatment of NSCLC in a population of patients each having NSCLC with the methods disclosed herein achieves an ORR of at least 56%. In certain embodiments, treatment of NSCLC in a population of patients each having NSCLC with the methods disclosed herein achieves an ORR of at least 69%. In certain embodiments, treatment of NSCLC in a population of patients each having NSCLC with the methods discloses herein achieves an ORR of about 44%. In certain embodiments, treatment of NSCLC in a population of patients each having NSCLC with the methods discloses herein achieves an ORR of 44%. In certain embodiments, treatment of NSCLC in a population of patients each having NSCLC with the methods disclosed herein achieves an ORR of about 56%. In certain embodiments, treatment of NSCLC in a population of patients each having NSCLC with the methods discloses herein achieves an ORR of 56%. In certain embodiments, treatment of a NSCLC in a population of patients each having NSCLC achieves an ORR of about 69%. In certain embodiments, treatment of NSCLC in a population of patients each having NSCLC with the methods discloses herein achieves an ORR of 69%.

In certain embodiments, treatment of NSCLC in a population of patients each having NSCLC and each having a PD-L1 TPS≥50% achieves an ORR of about 75%. In certain embodiments, treatment of NSCLC in a population of patients each having NSCLC and each having a PD-L1 TPS≥50% achieves an ORR of 75%. In certain embodiments, treatment of NSCLC in a population of patients having NSCLC and each having a PD-L1 TPS≥50% achieves an ORR of about 69% or greater.

In certain embodiments, treatment of NSCLC in a population of patients each having NSCLC and each having a PD-L1 TPS<50% achieves an ORR of about 44%. In certain embodiments, treatment of NSCLC in a population of patients each having NSCLC and each having a PD-L1 TPS<50% achieves an ORR of 44%. In certain embodiments, treatment of NSCLC in a population of patients having NSCLC and each having a PD-L1 TPS<50% achieves an ORR of about 44% or greater.

In certain embodiments, treatment of NSCLC in a population of patients each having NSCLC achieves an ORR of about 54%, irrespective of PD-L1 TPS status. In certain embodiments, treatment of NSCLC in a population of patients each having NSCLC achieves an ORR of 54%, irrespective of PD-L1 TPS status. In certain embodiments, treatment of NSCLC in a population of patients each having NSCLC achieves an ORR of about 54% or greater. In certain embodiments, treatment of NSCLC in a population of patients each having NSCLC achieves an ORR of about 56% or greater.

In certain embodiments, treatment of squamous metastatic NSCLC in a population of patients each having squamous metastatic NSCLC and each having a PD-L1 TPS≥50% achieves an ORR of about 73%. In certain embodiments, treatment of squamous metastatic NSCLC in a population of patients each having squamous metastatic NSCLC and each having a PD-L1 TPS≥ 50% achieves an ORR of 73%. In certain embodiments, treatment of squamous metastatic NSCLC in a population of patients having squamous metastatic NSCLC and each having a PD-L1 TPS≥50% achieves an ORR of about 73% or greater.

In certain embodiments, treatment of squamous metastatic NSCLC in a population of patients each having squamous metastatic NSCLC and each having a PD-L1 TPS<50% achieves an ORR of about 54%. In certain embodiments, treatment of squamous metastatic NSCLC in a population of patients each having squamous metastatic NSCLC and each having a PD-L1 TPS<50% achieves an ORR of 54%. In certain embodiments, treatment of squamous metastatic NSCLC in a population of patients having squamous metastatic NSCLC and each having a PD-L1 TPS<50% achieves an ORR of about 54% or greater.

In certain embodiments, treatment of NSCLC in a patient having a PD-L1 TPS of ≥50% achieves a partial response or a complete response. In certain embodiments, treatment of NSCLC in a patient having a PD-L1 TPS<50% achieves a partial response or a complete response. In certain embodiments, treatment of NSCLC in a patient achieves a partial response or a complete response. In certain embodiments, treatment of NSCLC in a patient having PD-L1 TPS of ≥50% achieves a partial response. In certain embodiments, treatment of NSCLC in a patient having PD-L1 TPS<50% achieves a partial response. In certain embodiments, treatment of NSCLC in a patient achieves a partial response. In certain embodiments, treatment of NSCLC in a patient achieves a>30% decrease in the sum of the LD of target lesions, taking as reference the baseline sum LD. In certain embodiments, treatment of NSCLC in a patient achieves stable disease. In certain embodiments, the partial response is about a 30% or greater reduction in the size of one or more of the measurable tumors from baseline. In certain embodiments, the partial response is about a 40% or greater reduction in the size of one or more of the measurable tumors from baseline. In certain embodiments, the partial response is about a 50% or greater reduction in the size of one or more of the measurable tumors from baseline. In certain embodiments, the partial response is about a 60% or greater reduction in the size of one or more measurable tumors from baseline. In certain embodiments, the partial response is measured from baseline to approximately 13 weeks or more from baseline.

In certain embodiments, when the methods disclosed herein are used to treat as population of patients having metastatic squamous metastatic NSCLC and a PD-L1 TPS≥50%, the partial response is about 64%-74%. In certain embodiments, when the methods disclosed herein are used to treat a population of patients having metastatic squamous metastatic NSCLC and a PD-L1 TPS<50%, the partial response is about 46%-54%. In certain embodiments, the partial response is measured from baseline to approximately 13 weeks or more from baseline.

In certain embodiments, when the methods disclosed herein are used to treat as population of patients having metastatic NSCLC and a PD-L1 TPS≥50%, the partial response is about 69% or greater. In certain embodiments, when the methods disclosed herein are used to treat a population of patients having metastatic NSCLC and a PD-L1 TPS<50%, the partial response is about 44% or greater. In certain embodiments, when the methods disclosed herein are used to treat a population of patients having metastatic NSCLC, the partial response is about 56% or greater.

In certain embodiments, treatment of NSCLC in a population of patients having metastatic NSCLC across all PD-L1 TPS scores achieves a disease control rate (DCR) of about 82% or greater after at least 6 weeks from baseline. In certain embodiments, treatment of NSCLC in a population of patients having metastatic NSCLC with the methods disclosed herein, across all PD-L1 TPS scores, achieves a disease control rate (DCR) of 81% after at least 6 weeks from baseline. In certain embodiments, treatment of NSCLC in a population of patients having metastatic NSCLC and PD-L1 tumor proportion scores of <50% achieves a disease control rate (DCR) of greater than or equal to about 78% after at least 6 weeks from baseline. In certain embodiments, treatment of NSCLC in a patient having metastatic NSCLC and a PD-L1 tumor proportion scores of <50% achieves a disease control rate (DCR) of 78% after at least 6 weeks from baseline. In certain embodiments, treatment of NSCLC in a population of patients having metastatic NSCLC and PD-L1 TPS scores of ≥50% achieves a DCR of about 86% or greater after at least 6 weeks from baseline. In certain embodiments, treatment of NSCLC in a population of patients having metastatic NSCLC and PD-L1 TPS scores of ≥50% achieves a DCR of 86% after at least 6 weeks from baseline.

In certain embodiments, treatment of squamous metastatic NSCLC in a population of patients having metastatic squamous metastatic NSCLC and PD-L1 tumor proportion scores of <50% achieves a disease control rate (DCR) of greater than or equal to about 82% after at least 6 weeks from baseline. In certain embodiments, treatment of squamous metastatic NSCLC in a patient having metastatic squamous metastatic NSCLC and a PD-L1 tumor proportion scores of <50% achieves a disease control rate (DCR) of 82% after at least 6 weeks from baseline. In certain embodiments, treatment of squamous metastatic NSCLC in a population of patients having metastatic squamous metastatic NSCLC and PD-L1 TPS scores of ≥50% achieves a DCR of about 85% or greater after at least 6 weeks from baseline. In certain embodiments, treatment of squamous metastatic NSCLC in a population of patients having metastatic squamous metastatic NSCLC and PD-L1 TPS scores of ≥ 50% achieves a DCR of 85% after at least 6 weeks from baseline.

In certain embodiments, treatment of NSCLC in a population of patients having metastatic NSCLC and PD-L1 tumor proportion scores of <50% with the methods disclosed herein achieves a duration of response (DOR) of about 88% at about 6 months from baseline. In certain embodiments, treatment of NSCLC in a population of patients having metastatic NSCLC and PD-L1 tumor proportion scores of <50% achieves a duration of response (DOR) of 88% at about 6 months from baseline. In certain embodiments, treatment of NSCLC in a population of patients having metastatic NSCLC and PD-L1 TPS of ≥50% with the methods disclosed herein achieves a DOR of about 88% at about 6 months from baseline. In certain embodiments, treatment of NSCLC in a population of patients having metastatic NSCLC and PD-L1 TPS of ≥50% with the methods disclosed herein achieves a DOR of 88% at about 6 months from baseline. In certain embodiments, treatment of NSCLC in a population of patients having metastatic NSCLC with the methods disclosed herein achieves a DOR of about 87% across all PD-L1 TPS scores about 6 months from baseline. In certain embodiments, treatment of NSCLC in a population of patients having metastatic NSCLC with the methods disclosed herein achieves a DOR of 87% across all PD-L1 TPS scores about 6 months from baseline.

In certain embodiments, treatment of NSCLC in a population of patients having metastatic NSCLC with the methods disclosed herein achieves/results in less than about 10% serious treatment emergent adverse effects (TEAEs). In certain embodiments, treatment of NSCLC in a population of patients having metastatic NSCLC with the methods disclosed herein achieves/results in less than about 9% serious TEAEs.

In certain embodiments, treatment of NSCLC in a population of patients having metastatic NSCLC achieves less than about 25% TEAEs of grade≥3. In certain embodiments, treatment of NSCLC in a population of patients having metastatic NSCLC achieves/results in less than about 24% TEAEs of grade≥3.

In certain embodiments, the TEAE is interstitial lung disease (ILD). In certain embodiments, less than about 10% of the patients treated with the methods disclosed herein were observed to have ILD. In certain embodiments, less than about 9% of the patients treated with the methods disclosed herein were observed to have ILD. In certain embodiments, less than about 5% of the patients treated with the methods disclosed herein were observed to have ILD. In certain embodiments, ILD was not observed in the patients treated with the methods disclosed herein.

### EXAMPLES

### Example 1

### Ph 1/2 IMMU-132-01 Basket Trial (NCT01631552)

SG (as a monotherapy) was evaluated in metastatic NSCLC in a clinical trial: IMMU-132-01 (completed, 54 patients enrolled). The ORR based on local response assessment was 16.7% for the NSCLC population; all responses were PRs. The median duration of response (DOR) by local assessment was 6.0 months. The median progression free survival was 5.2 months, and the median overall survival was 9.5 months.

Table 5 shows the key efficacy results for patients having NSCLC in the IMM-132-01 basket trial.

TABLE 5
Summary of Responses for Assessable Patients With an Intention-
to-Treat Analysis for PFS and OS

| Response | No. (%) |
|---|---|
| All patients | |
| Best overall response (n = 47) | |
| PR | 9 (19) |
| SD | 23 (49) |
| PD | 15 (32) |
| Objective response duration, months | |
| Median (95% CI) | 6.0 (4.8 to 8.3) |
| Clinical benefit (PR + SD ≥4 months) | 20 (43) |
| PFS (n = 54), months | |
| Median (95% CI) | 5.2 (3.2 to 7.1) |
| OS (n = 54), months | |
| Median (95% CI) | 9.5 (5.9 to 16.7) |
| Patients with prior CPI therapy (n = 14) | |
| Best overall response | |
| PR | 2 (14) |
| SD | 7 (50) |
| PD | 5 (36) |
| Clinical benefit (PR + SD ≥4 months) | 5 (36) |
| PFS, months | |
| Median (95% CI) | 5.2 (2.0 to 5.5) |
| OS, months | |
| Median (95% CI) | 14.6 (5.9 to 14.6) |

Abbreviations:
OS, overall survival;
PD, progressive disease;
PFS, progression-free survival;
PR, partial response
SD, stable disease.

### Example 2

### Phase 2 Clinical Trial-EVOKE-02 (NCT05186974)

Adults with previously untreated metastatic NSCLC, no actionable genomic alterations, and an ECOG performance status of 0 or 1 were enrolled in Cohorts A and B. Patients with PD-L1 tumor proportion score≥50% (Cohort A) and PD-L1 TPS<50% (Cohort B) received SG 10 mg/kg on Day 1 and 8 and pembrolizumab 200 mg on Day 1 of a 21-day cycle. The trial endpoints included objective response rate (ORR; per RECIST v1.1), progression-free survival, duration of response, disease control rate, overall survival, and safety. Safety results were reported in safety-evaluable patients who received≥1 dose of study treatment and efficacy results were reported in efficacy-evaluable patients with ≥13.0 weeks of follow-up.

### Inclusion Criteria

Patients met all of the inclusion criteria outlined below at screening/Day −1 were eligible for participation in this study, with no waivers for patient eligibility offered or permitted:

The patients were 18 years of age or older and able to understand and give written informed consent.

The patients had a life expectancy≥3 months.

The patients had pathologically documented NSCLC. The pathologically documented NSCLC was by way of stage IV NSCLC at the time of enrollment based on the American Joint Committee on Cancer, Eighth Edition; negative test results for EGFR and ALK; no known actionable genomic alterations in ROS1, NTRK, BRAF, RET mutations, or other actionable driver oncogenes with approved therapies for frontline treatment; and have provided tumor tissue from locations not radiated prior to biopsy. If status of EGFR and ALK, or ROS1, NTRK, BRAF, RET and other actionable driver oncogenes was unknown, tumor tissue testing was required. Formalin-fixed specimens after the patient had been diagnosed with metastatic disease were preferred for evaluation of Trop-2 expression and determination of PD-L1 status prior to enrollment if not already performed by an approved 22C3 assay. Biopsies obtained prior to receipt of adjuvant/neoadjuvant chemotherapy were permitted if a recent biopsy was not feasible. Bone biopsies and fine-needle aspirations were not accepted as suitable tissues. If no tissue is available, a new biopsy was obtained prior to enrollment to the study.

The patients had measurable disease by CT or magnetic resonance imaging (MRI) as per RECIST Version 1.1 criteria by investigator. Tumor lesions situated in a previously irradiated area were considered measurable if progression had been demonstrated in such lesions. Historical images within 28 days of the screening visit were accepted as a screening image if deemed acceptable in the opinion of the investigator.

The patients had no prior systemic treatment for metastatic Non-Small Cell Lung Cancer (mNSCLC). Patients who received adjuvant or neoadjuvant therapy were eligible if the adjuvant/neoadjuvant therapy was completed at least 6 months prior to the development of metastatic disease for a platinum agent.

The patients had an Eastern Cooperative Oncology Group (ECOG) performance status score of 0 or 1 assessed within 7 days prior to treatment.

The patients had adequate hematologic counts without transfusional or growth factor support within 10 days of study drug initiation (hemoglobin≥9 g/dL, absolute neutrophil count [ANC]≥1500/mm³, and platelets≥100,000/μL). Hemoglobin criteria was required to be met without packed red blood cell (pRBC) transfusion within the prior 2 weeks. Patients could be on a stable dose of erythropoietin (≥approximately 3 months).

The patients had adequate hepatic function (bilirubin≤1.5×ULN, AST and ALT≤2.5×ULN or ≤5×ULN if known liver metastases, and serum albumin>3 g/dL).

The patients had creatinine clearance of at least 30 mL/min as assessed by the Cockcroft-Gault equation (Cockcroft 1976). For patients assigned to cohorts with cisplatin, creatinine clearance must be at least 60 mL/min.

Patients with HIV had to be on antiretroviral therapy (ART) and have a well-controlled HIV infection/disease defined as: (1) patients on ART with a CD4+ T-cell count>350 cells/mm³ at time of screening; (2) patients on ART that achieved and maintained virologic suppression defined as confirmed HIV RNA level below 50 copies/mL or the lower limit of qualification (below the limit of detection) using the locally available assay at the time of screening and for at least 12 weeks prior to screening; (3) patients on ART on a stable regimen, without changes in drugs or dose modification, for at least 4 weeks prior to study entry; and (4) the combination ART regimen must not contain any medications that may interfere with SN-38 metabolism.

Finally, male patients and female patients of childbearing potential who engage in heterosexual intercourse must have agreed to use protocol-specified method(s) of contraception.

### Exclusion Criteria

Patients who met any of the exclusion criteria outlined below at screening/Day −1 were not eligible to be enrolled in this study, with no waivers for patient eligibility will be offered or permitted:

The patients had mixed small-cell lung cancer (SCLC) and NSCLC histology.

The patients had an active second malignancy. Patients with a history of malignancy who had been completely treated, with no evidence of cancer for 3 years prior to enrollment, or patients with surgically cured tumors with low risk of recurrence (e.g., nonmelanoma skin cancer, histologically-confirmed complete excision of carcinoma in situ, or similar) were allowed to enroll.

The patients had NSCLC that was eligible for definitive local therapy alone.

The patients had a diagnosis of immunodeficiency or were receiving chronic systemic steroid therapy (in dosing exceeding 10 mg daily of prednisone equivalent) or any other form of immunosuppressive therapy within 7 days prior to the first dose of study drug. Intermittent use of topical, inhalational, intranasal, and intraocular steroids was permitted.

The patients had an active autoimmune disease that has required systemic treatment in past 2 years (i.e., with use of disease-modifying agents, corticosteroids, or immunosuppressive drugs). Replacement therapy (e.g., thyroxine, insulin, or physiologic corticosteroid replacement therapy for adrenal or pituitary insufficiency) was not considered a form of systemic treatment and was allowed.

The patients had an allogenic tissue or solid organ transplant.

The patients had severe (≥Grade 3) hypersensitivity to SG, pembrolizumab, carboplatin, or cisplatin, their metabolites, or formulation excipient.

The patients had a requirement for ongoing therapy with or prior use of any prohibited medications.

The patient had received prior therapy with an anti-PD-1, anti-PD-L1, or anti-PD-L2 agent or with an agent directed to another stimulatory or coinhibitory T-cell receptor (e.g., CTLA-4, OX 40, CD137), and were discontinued from that treatment due to a Grade 3 or higher immune-related AEs (irAE).

The patients had received radiation therapy to the lung that is >30 Gy within 6 months of the first treatment cycle.

The patients could not have received systemic anticancer treatment within the previous 6 months or radiation therapy within 2 weeks, or stereotactic radiosurgery within 72 hours prior to enrollment. Patients must have recovered (i.e., >Grade 2 was considered not recovered) from AEs at the time of study entry. Patients with any grade alopecia were an exception to this criterion and qualified for the study. If patients received major surgery, they must have recovered adequately from the toxicity and/or complications from the intervention prior to starting therapy. Patients must have recovered from all radiation-related toxicities, not have required corticosteroids, and not have had radiation pneumonitis. A 1-week washout was permitted for palliative radiation (≤2 weeks of radiotherapy) to non-central nervous system (CNS) disease.

The patients had previously received treatment with any of the following: (1) topoisomerase 1 inhibitors, or any agent including an ADC containing a chemotherapeutic agent targeting topoisomerase 1; or (2) Trop-2-targeted therapy.

The patients were currently participating in or had participated in a study of an investigational agent or had used an investigational device within 4 weeks prior to the first dose of study treatment. Patients who had entered the follow-up phase of an investigational study could participate as long as it had been 4 weeks after the last dose of the previous investigational agent.

The patients had clinically severe pulmonary compromise resulting from intercurrent pulmonary illnesses including, but not limited to, any underlying pulmonary disorder (i.e., pulmonary emboli within 3 months of enrollment, severe asthma, severe chronic obstructive pulmonary disease (COPD), restrictive lung disease, pleural effusion, etc.); any autoimmune, connective tissue, or inflammatory disorders with pulmonary involvement (i.e., rheumatoid arthritis, Sjogren syndrome, sarcoidosis, etc.); or prior pneumonectomy.

The patients had known active CNS metastases and/or carcinomatous meningitis. Patients with previously treated brain metastases could participate provided they had stable CNS disease for at least 4 weeks prior to enrollment and all neurologic symptoms have returned to baseline, had no evidence of new or enlarging brain metastases, and were taking≤10 mg/day of prednisone or its equivalent. All patients with carcinomatous meningitis were excluded regardless of clinical stability. Patients were clinically stable per investigator's assessment and are taking≤10 mg/day of prednisone or its equivalent for at least 14 days prior to first dose of study treatment.

The patients had active chronic inflammatory bowel disease (ulcerative colitis, Crohn's disease) or gastrointestinal (GI) perforation within 6 months of enrollment.

The patients had a history of (noninfectious) pneumonitis/interstitial lung disease that required steroids or had current pneumonitis/interstitial lung disease.

The patients had active serious infection requiring antibiotics.

The patients were positive for HIV-1 or 2 with a history of Kaposi sarcoma and/or multicentric Castleman disease.

The patients had active or chronic hepatitis B infection, or were positive for hepatitis B surface antigen. Patients who tested positive for hepatitis B core antibody required hepatitis B virus DNA by quantitative polymerase chain reaction for confirmation of active disease.

The patients were positive for hepatitis C antibody and had detectable hepatitis C virus (HCV) viral load.

The patients were positive for a serum pregnancy test or were women who were lactating.

The patients had other concurrent medical or psychiatric conditions that, in the investigator's opinion, were likely to confound study interpretation or prevent completion of study procedures and follow-up examinations.

The patients received a live-virus vaccination within 30 days of planned treatment start. Seasonal flu vaccines that did not contain live virus were permitted.

### Results

As of 13 Jan. 2023, 44 patients (Cohort A, n=16; Cohort B, n=28) were enrolled and received SG+pembrolizumab. Median age was 68 years (range, 47-80); 64% of patients had non-squamous histology and 77% had an ECOG performance status of 1. In efficacy-evaluable patients (Cohort A, n=8; Cohort B, n=18), ORR by investigator assessment was 75% (5 confirmed partial responses [PRs] and 1 PR pending confirmation) in Cohort A; and 44% (7 confirmed PRs and 1 PR pending confirmation) in Cohort B (as shown in Table 6).

TABLE 6
Efficacy Results

| | Cohort A | Cohort B | |
|---|---|---|---|
| | PD-L1 | PD-L1 | |
| | TPS ≥50% | TPS <50% | Total |
| | SG + Pembro | SG + Pembro | SG + Pembro |
| Efficacy by INV | n = 29 | n = 32 | N = 61 |
| ORR (95% CI), % | 69 (49-85) | 44 (26-62) | 56 (42-69) |
| BOR, n (%) | | | |
| CR | 0 | 0 | 0 |
| PR | 20^{a} (69) | 14^{b} (44) | 34 (56) |
| SD | 5 (17) | 11 (34) | 16 (26) |
| PD | 3 (10) | 2 (6) | 5 (8) |
| NAC | 1 (3) | 5 (16) | 6 (10) |
| DCR (95% CI), % | 86 (68-96) | 78 (60-91) | 82 (70-91) |
| Median DOR (95% | NR (5.6-NR) | NR (3.5-NR) | NR (7.9-NR) |
| CI), months | | | |
| DOR rate at 6 months | 88 (39-98) | 88 (39-98) | 87 (58-97) |
| (95% CI)4, % | | | |

^{a}18 confirmed PRs and 2 PR pending confirmation;
^{b}12 confirmed PRs and 2 PR pending confirmation;
^{c}5 patients died prior to first scheduled tumor assessment (Cohort A, n = 1; Cohort B, n = 4), and one patient from Cohort B withdrew consent;
devaluated in patients with confirmed CR or PR, and based on Kaplan-Meier estimates.
BOR, best overall response;
CI, confidence interval;
CR, complete response;
DCR, disease control rate;
DOR, duration of response;
INV, investigator assessment;
NA, not assessed;
ORR, objective response rate;
PR, partial response;
PD, progressive disease;
SD, stable disease.
The ORR was defined as BOR of CR and PR.
The DCR was defined as BOR of CR, PR and SD at six weeks or more.

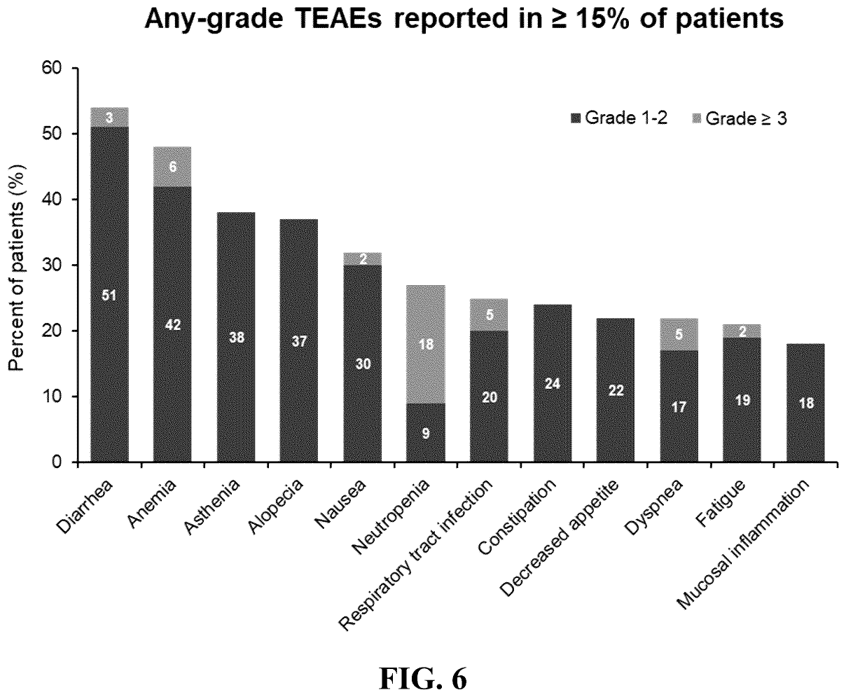

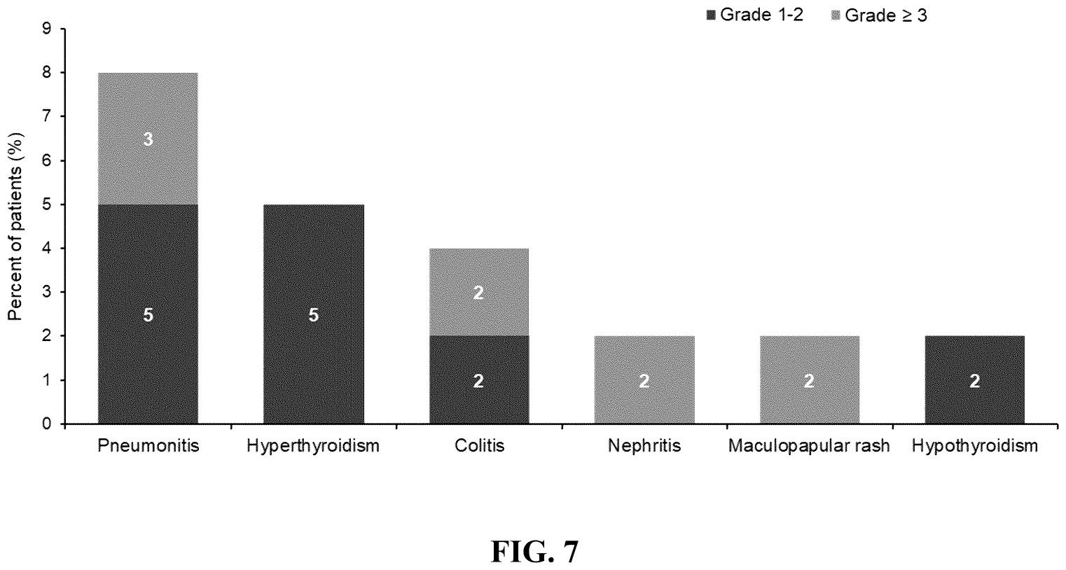

Among safety-evaluable patients (n=63), the incidence of any-grade treatment-emergent adverse events (TEAEs) was 100% (grade 3/4, 70%; Table 7). As shown in FIG. 6, the most common any-grade TEAEs were diarrhea (86%), anemia (76%), asthenia (60%), and alopecia (59%). The most common immune-mediated TEAEs were pneumonitis and hyperthyroidism (13% and 8%, respectively; FIG. 7). Grade 3 pneumonitis was the highest grade observed, and was observed in 2 (3%) patients. TEAEs leading to discontinuation of SG occurred in 18% of patients. TEAEs leading to death were reported in 4 (6%) patients, and only 1 (2%) was considered related to study treatment.

TABLE 7
Safety Results

| | Total Patients |
|---|---|
| | SG + Pembro |
| Safety-evaluable patients, n (%) | n = 63 |
| Any-grade TEAEs | 63 (100) |
| Related to study treatment | 57 (90) |
| Grade ≥3 TEAEs | 44 (70) |
| Related to study treatment | 24 (38) |
| Serious TEAEs | 34 (54) |
| Related to study treatment | 9 (14) |
| TEAEs leading to treatment discontinuation | 11 (18) |
| TEAEs leading to discontinuation of SG | 9 (14) |
| TEAEs leading to SG dose reductions | 11 (18) |
| TEAEs leading to deatha | 4 (6) |
| Related to study treatment | 1 (2) |

### Table 8 Efficacy Results

Efficacy results by investigator assessment at data cutoff (Jun. 16, 2023) is shown in Table 8. At this data cutoff, the median (range) follow-up for Cohorts A and B was 5.0 (1.7-12.0) and 5.8 (1.0-12.2) months, respectively.

TABLE 8
Efficacy Results

| | Cohort A | Cohort B | |
|---|---|---|---|
| | PD-L1 | PD-L1 | |
| | TPS ≥50% | TPS <50% | Total |
| | SG + Pembro | SG + Pembro | SG + Pembro |
| Efficacy by INV* | n = 30 | n = 33 | N = 61 |
| ORR (95% CI), % | 69 (49-85) | 44 (26-62) | 56 (52-69) |
| PR, n confirmed and | 20 (69) | 14 (44) | 34 (56) |
| unconfirmed, n (%) | | | |
| Confirmed PR, n (%) | 18 (62) | 12 (38) | 30 (49) |
| SD | 5 (17) | 11 (34) | 16 (26) |
| PD | 3 (10) | 2 (6) | 5 (8) |
| DCR (95% CI), % | 86 (68-96) | 78 (60-91) | 82 (70-91) |
| DOR rate at 6 months | 88 (39-98) | 88 (39-98) | 87 58-97) |
| (95% CI)^{d}, % | | | |

*INV, investigator assessment;
NA, not assessed;
ORR, objective response rate;
PR, partial response;
PD, progressive disease;
SD, stable disease.
The ORR was defined as BOR of CR and PR.
The DCR was defined as BOR of CR, PR and SD at six weeks or more.

### Table 9 Cohort B PD-L1 TPS Subgroup Analysis

Table 9 shows efficacy results for Cohort B at data cutoff (Jun. 16, 2023).

TABLE 9
Efficacy Results.

| | Cohort B PD-L1 TPS | Cohort B PD-L1 |
|---|---|---|
| | 1%-49% Subgroup | TPS <1% Subgroup |
| Efficacy by INV | n = 15 | n = 17 |
| ORR, % (95% CI) | 53 (27%-79%) | 35 (14%-62%) |
| PR, confirmed and | 8 (53) | 6 (35) |
| unconfirmed, n (%) | | |
| Confirmed PR, n (%) | 7 (47) | 5 (29) |
| SD, n (%) | 7 (47) | 4 (24) |
| PD, n (%) | 0 | 2 (12) |
| DCR, % (95% CI) | 100 (78%-00%) | 59 (33%-82%) |

ORR is BOR of CR+PR. DCR is BOR of CR+PR+SD≥6 weeks. BOR is best overall response, DCR is disease control rate, DOR is duration of response, and SD is stable disease. Other acronyms are defined herein.

Table 10 shows the response rates for frontline SG+pembro amongst patients with squamous and non-squamous mNSCLC.

In Cohort A, the ORR was 72.7% (8/11) in patients with squamous mNSCLC and 66.7% (12/18) in patients with nonsquamous mNSCLC (Table 10). In Cohort B, the ORR was 53.8% (7/13) and 36.8% (7/19), respectively, in patients with squamous and nonsquamous mNSCLC (Table 10). Median DOR was not reached in either cohort.

TABLE 10
Efficacy Results.

| | Squamous mNSCLC | | Non-squamous mNSCLC | |
|---|---|---|---|---|
| | Cohort A | Cohort B | Cohort A | Cohort B |
| | PD-L1 | PD-L1 | PD-L1 | PD-L1 |
| | TPS ≥50% | TPS <50% | TPS ≥50% | TPS <50% |
| Efficacy by Investigator assessment | (n = 11) | (n = 13) | (n = 18) | (n = 19) |
| ORR, n (%) | 8 (72.7) | 7 (53.8) | 12 (66.7) | 7 (36.8) |
| 95% CI | 39.0-94.0 | 25.1-80.8 | 41.0-86.7 | 16.3-61.6 |
| Best overall response, n (%) | | | | |
| PR | 8 (72.7) | 7 (53.8) | 12 (66.7) | 7 (36.8) |
| Confirmed PR | 7 (63.6) | 6 (46.2) | 11 (61.1) | 6 (31.6) |
| SD | 1 (9.1) | 4 (30.8) | 4 (22.2) | 7 (36.8) |
| PD | 2 (18.2) | 0 | 1 (5.6) | 2 (10.5) |
| Not assessed | 0 | 2 (15.4) | 1 (5.6) | 3 (15.8) |
| DCR, n (%) | 9 (81.8) | 11 (84.6) | 16 (88.9) | 14 (73.7) |
| [95% CI] | [48.2-97.7] | [54.6-98.1] | [65.3-98.6] | [48.8-90.9] |

ORR, objective response rate;
PR, partial response;
PD, progressive disease;
SD, stable disease;
CI, confidence interval.
ORR is based on unconfirmed responses per RECIST v1.1, except where otherwise stated. The best overall response represents complete response + PR + SD (for ≥6 weeks if confirmed).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of treating a metastatic non-small cell lung cancer (NSCLC) in a patient, wherein the patient has not had a prior systemic therapy for metastatic NSCLC, comprising co-administering:
a. 10 mg/kg of an antibody-drug conjugate (ADC) comprising an anti-Trop-2 antibody or an antigen-binding fragment thereof as an intravenous infusion on day 1 and day 8 of one or more 21-day treatment cycles, wherein the anti-Trop-2 antibody or the antigen binding fragment thereof comprises light chain complementarity determining regions (CDRs) comprising a sequence of amino acids as set forth in SEQ ID NOs: 13, 14 and 15 and heavy chain CDRs comprising a sequence of amino acids as set forth in SEQ ID NOs: 16, 17 and 18, and wherein the ADC has a formula MAb-CL2A-SN-38, with the formula represented by: 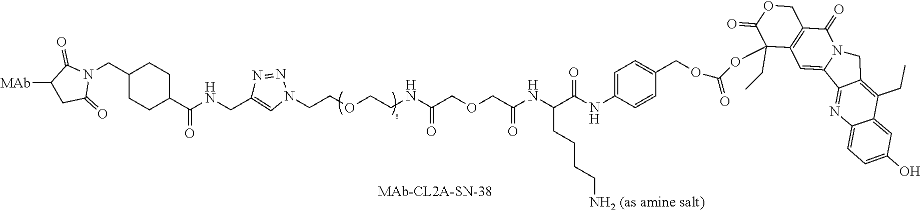
and
b. 200 mg of an anti-PD-1 antibody or an antigen-binding fragment thereof as an intravenous infusion on day 1 of the one or more 21-day treatment cycles, wherein the anti-PD-1 antibody or the antigen-binding fragment thereof comprises light chain CDRs comprising a sequence of amino acids as set forth in SEQ ID NOs: 3, 4 and 5 and heavy chain CDRs comprising a sequence of amino acids as set forth in SEQ ID NOs: 8, 9 and 10; and wherein the method achieves a complete response or a partial response in the patient.

2. (canceled)

3. The method of claim 1, wherein the ADC is sacituzumab govitecan (SG).

4.-6. (canceled)

7. The method of claim 1, wherein the anti-PD-1 antibody or the antigen fragment thereof is pembrolizumab.

8. The method of claim 1, wherein the ADC is SG and the anti-PD-1 antibody or the antigen fragment thereof is pembrolizumab.

9. The method of claim 1, wherein the patient has a PD-L1 tumor proportion score (TPS) of ≥50%.

10. The method of claim 1, wherein the patient has a PD-L1 TPS of <50%.

11. The method of claim 1, wherein the patient has non-squamous NSCLC.

12. The method of claim 1, wherein the patient has squamous NSCLC.

13. The method of claim 1, wherein the method does not comprise concomitant administration of chemotherapy.

14. The method of claim 13, wherein the method does not comprise concomitant administration of platinum-based chemotherapy.

15. The method of claim 1, wherein the method does not comprise concomitant administration of an anti-TGIT antibody.

16. The method of claim 8, wherein the partial response is about 50% or greater reduction in a size of one or more measurable tumors from baseline to approximately 13 weeks or more from baseline.

17. The method of claim 16, wherein the partial response is about 60% or greater reduction in the size of one or more of the measurable tumors from baseline to approximately 13 weeks or more from baseline.

18. The method of claim 8, wherein when the method is used to treat metastatic NSCLC in a population of patients with a PD-L1 TPS of ≥50%, the method achieves a partial response rate of about 69% or greater.

19. The method of claim 8, wherein when the method is used to treat metastatic NSCLC in a population of patients with a PD-L1 TPS of <50%, the method achieves a partial response rate of about 44% or greater.

20. The method of claim 8, wherein when the method is used to treat metastatic NSCLC in a population of patients, the method achieves a partial response of about 56% or greater irrespective of the population of patient's PD-L1 TPS status.

21. A method of treating a population of patients having metastatic NSCLC without concomitant administration of chemotherapy comprising co-administering:
a. 10 mg/kg of an antibody-drug conjugate (ADC) comprising an anti-Trop-2 antibody or an antigen-binding fragment thereof as an intravenous infusion on day 1 and day 8 of one or more 21-day treatment cycles, wherein the anti-Trop-2 antibody or the antigen binding fragment thereof comprises light chain complementarity determining regions (CDRs) comprising a sequence of amino acids as set forth in SEQ ID NOs: 13, 14 and 15 and heavy chain CDRs comprising a sequence of amino acids as set forth in SEQ ID NOs: 16, 17 and 18, and wherein the ADC has a formula MAb-CL2A-SN-38, with the formula represented by: 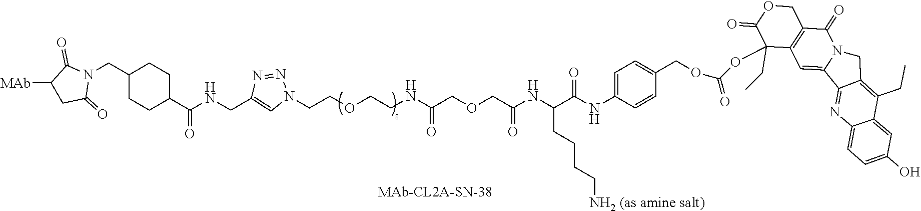
and
b. 200 mg of an anti-PD-1 antibody or an antigen-binding fragment thereof as an intravenous infusion on day 1 of the one or more 21-day treatment cycles, wherein the anti-PD-1 antibody or the antigen-binding fragment thereof comprises light CDRs comprising a sequence of amino acids as set forth in SEQ ID NOs: 3, 4 and 5 and heavy chain CDRs comprising a sequence of amino acids as set forth in SEQ ID NOs: 8, 9 and 10; wherein the population of patients have not received prior systemic therapy for metastatic NSCLC.

22. (canceled)

23. The method of claim 21, wherein the ADC is SG.

24.-26. (canceled)

27. The method of claim 21, wherein the anti-PD-1 antibody or the antigen-binding fragment thereof is pembrolizumab.

28. (canceled)

29. The method of claim 21, wherein the population of patients has a PD-L1 TPS of ≥50%.

30. The method of claim 21, wherein the population of patients has a PD-L1 TPS of <50%.

31. The method of claim 21, wherein the population of patients has non-squamous NSCLC.

32. The method of claim 21, wherein the population of patients has squamous NSCLC.

33. The method of claim 21, wherein the treatment comprises achieving of clinical efficacy.

34. The method of claim 29, wherein the ORR is about 69%.

35. The method of claim 30, wherein the ORR is about 44%.

36. The method of claim 29, wherein the DCR is about 86%.

37. The method of claim 30, wherein the DCR is about 78%.

38. The method of claim 29, wherein the DOR at about 6 months from baseline is about 88%.

39. The method of claim 30, wherein the DOR at about 6 months from baseline is about 88%.

40. The method of claim 21, wherein the method achieves less than about 10% serious treatment emergent adverse events (TEAEs).

41. The method of claim 40, wherein the method achieves less than about 9% serious TEAEs.

42. The method of claim 21, wherein the method achieves less than about 25% TEAEs of grade≥3.

43. The method of claim 42, wherein the method achieves less than about 24% TEAEs of grade≥3.

44. (canceled)

45. The method of claim 21, wherein a TEAE comprises interstitial lung disease (ILD).

46. The method of claim 45, wherein ILD is observed in less than about 10% of the patients.

47. The method of claim 46, wherein ILD is observed in less than about 9% of the patients.

48. The method of claim 47, wherein ILD is observed in less than about 5% of the patients.

49. The method of claim 28, wherein ILD is not observed in the patients.

50. A method of improving clinical efficacy compared to administration of an anti-PD-L1 antibody monotherapy or compared to administration of an antibody drug conjugate monotherapy, in a population of patients having metastatic non-squamous or squamous NSCLC, without concomitant administration of a chemotherapy, comprising co-administering:
a. 10 mg/kg SG as an intravenous infusion on day 1 and day 8 of one or more 21-day treatment cycles; and
b. 200 mg pembrolizumab on day 1 of the one or more 21-day treatment cycles;
wherein the population of patients have not received prior systemic therapy for metastatic NSCLC.

51. The method of claim 50, wherein clinical efficacy is improved irrespective of the population of patient's PD-L1 status.

52.-53. (canceled)

54. The method of claim 50, wherein the anti-PD-L1 antibody monotherapy is pembrolizumab.

55. The method of claim 50, wherein the antibody drug conjugate monotherapy is SG.

56. The method of claim 50, wherein the method does not comprise determination of a PD-L1 TPS status in the population of patients.

57. (canceled)

58. The method of claim 50, wherein the method does not include administration of an additional targeted oncology drug.

59. A method of achieving clinical efficacy in a patient having metastatic NSCLC irrespective of the patient's PD-L1 tumor proportion score, irrespective of the patient's NSCLC histology, without the concomitant administration of chemotherapy, and wherein the patient has not received prior systemic therapy for metastatic NSCLC, comprising co-administering:
a. 10 mg/kg of SG on day 1 and day 8 of one or more 21-day treatment cycles; and
b. 200 mg of pembrolizumab on day 1 of the one or more 21-day treatment cycles.

60.-62. (canceled)

63. The method of claim 8, wherein the partial response is about 30% or greater reduction in the size of one or more measurable tumors from baseline to approximately 13 weeks or more from baseline.

64. The method of claim 63, wherein the partial response is about 40% or greater reduction in the size of one or more measurable tumors from baseline to approximately 13 weeks or more from baseline.

65. The method of claim 10, wherein the patient has a PD-L1 TPS of 1%-49%.

66. The method of claim 10, wherein the patient has a PD-L1 TPS of <1%.

67. The method of claim 66, wherein the patient achieves a partial response, and wherein the partial response is about 50% or greater reduction in a size of one or more measurable tumors from baseline to approximately 13 weeks or more from baseline.

68. The method of claim 67, wherein the partial response is about 60% or greater reduction in a size of one or more measurable tumors from baseline to approximately 13 weeks or more from baseline.

69. The method of claim 65, wherein when the method is used to treat metastatic NSCLC in a population of patients with a PD-L1 TPS of 1%-49%, the method achieves an ORR of about 53%.

70. The method of claim 66, wherein when the method is used to treat metastatic NSCLC in a population of patients with a PD-L1 TPS of <1%, the method achieves an ORR of about 35%.

71. The method of claim 69, wherein the method achieves a partial response of about 47%-53%.

72. The method of claim 70, wherein the method achieves a partial response of about 29%-35%.

73.-102. (canceled)


SEQUENCE LISTING (20 sequences)

SEQ ID NO: 1 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 15 aa, synthetic construct
RASKGVSTSGYSYLH

SEQ ID NO: 4 - protein, 7 aa, synthetic construct
LASYLES

SEQ ID NO: 5 - protein, 9 aa, synthetic construct
QHSRDLPLT

SEQ ID NO: 6 - protein, 111 aa, synthetic construct [sequence omitted]

SEQ ID NO: 7 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 5 aa, synthetic construct
NYYMY

SEQ ID NO: 9 - protein, 17 aa, synthetic construct
GINPSNGGTNFNEKFKN

SEQ ID NO: 10 - protein, 11 aa, synthetic construct
RDYRFDMGFDY

SEQ ID NO: 11 - protein, 120 aa, synthetic construct [sequence omitted]

SEQ ID NO: 12 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 5 aa, synthetic construct
NYGMN

SEQ ID NO: 14 - protein, 17 aa, synthetic construct
WINTYTGEPTYTDDFKG

SEQ ID NO: 15 - protein, 12 aa, synthetic construct
GGFGSSYWYFDV

SEQ ID NO: 16 - protein, 11 aa, synthetic construct
KASQDVSIAVA

SEQ ID NO: 17 - protein, 7 aa, synthetic construct
SASYRYT

SEQ ID NO: 18 - protein, 9 aa, synthetic construct
QQHYITPLT

SEQ ID NO: 19 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 20 - protein, 108 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKR